(19) United States
(12) Patent Application Publication
(10) Pub. No.: US 2010/0247562 A1
(43) Pub. Date: Sep. 30, 2010

(54) Complexes Derived from Heterohybrid Cells and Uses Thereof

(75) Inventors: Jianlin Gong, Wayland, MA (US); Stuart K Calderwood, Chestnut Hill, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/438,685

(22) PCT Filed: Aug. 24, 2007
(86) PCT No.: PCT/US07/18719

§ 371 Date: Feb. 24, 2009

Related U.S. Application Data
(60) Provisional application No. 60/839,917, filed on Aug. 24, 2006.

(51) Int. Cl. A61K 39/385; C07K 14/435; A61P 35/00; A61K 39/00; A61K 38/17

(52) U.S. Cl. 424/193.1; 530/324; 424/184.1; 514/19.3

(57) ABSTRACT

The present invention relates, generally, to pharmaceutical compositions comprising Heat Shock Proteins (HSPs) and HSP complexes recovered from heterohybrid cells that are generated from the fusion of a first type of cell (Type I) with a second type of cell (Type II). In further embodiments, the present invention relates to preparing HSPs and/or HSP complexes recovered from heterohybrid, and to methods to treat and/or prevent diseases, such as cancer and infectious diseases by administering a pharmaceutical composition comprising a HSP and/or HSP complex recovered from heterohybrid cells that are generated from the fusion of a first type of cell (Type I) with a second type of cell (Type II).

### CROSS REFERENCE TO RELATED APPLICATIONS

This application is a 371 National Phase Entry Application of co-pending International Application PCT/US2007/0187719 filed Aug. 24, 2007, which designated the U.S., and claims the benefit under 35 U.S.C 119(e) of U.S. Provisional Patent Application Ser. No. 60/839,917 filed Aug. 24, 2006, the contents of which are incorporated herein by reference in their entirety.

### GOVERNMENT SUPPORT

The present application was supported by the National Institutes for Health (H1H) Grant Number CA87057 and the Government of the United States has certain rights thereto.

### FIELD OF THE INVENTION

The present invention relates generally to methods of immune stimulation and complexes that can be used.

More particularly, the present invention relates to using a sample of a population of heterohybrid cells. It also relates to methods for the production of such a cell population and uses thereof.

### BACKGROUND

Immunotherapy and vaccination has been used in treating and preventing, and in some cases eradicating diseases, such as polio, tetanus, tuberculosis, chicken pox, measles, hepatitis etc. Vaccinations with materials such as proteins can act as antigens to elicit an antigenic response and the induction of an antibody response or CD4⁺ helper T-cell response. Vaccinations or infection with live cells or infectious viruses can lead to a CD8⁺ cytotoxic T-lymphocyte (CTL) response, which is crucial for protection against cancers, infectious agents, infectious viruses and certain bacteria. Numerous studies have investigated methods to induce a CTL without using live agents. Heat shock protein complexes have been proposed as agents against cancers and infectious agents (Srivastava et al., (1994) Curr. Op. Immu. 6:728; Srivastava (1993) Adv. Cancer Res. 62:153).

Heat-shock proteins (HSP) play an important role as intracellular molecular chaperones in the pathways of protein folding in the cell (1,2). They play a role in the immune response when released from cells in complex with chaperoned antigenic peptides. When antigenic peptides are complexed with HSP, either (i) non-covalently associated with the substrate binding domain or (ii) when covalently bound to HSP70, and injected into mice, they can prime cytotoxic T lymphocyte (CTL) responses (3-12). Immunization with HSP-peptide complexes purified from cancer cells has been reported to provide protection against tumors derived from the cancer cells from which the HSP are purified (13-17). Moreover, treatment of mice bearing established cancers or residual tumors with such vaccines has been discussed in reducing the tumor burden and metastasis and in prolonging survival (16,18). In a number of clinical trials, immunization of patients with advanced malignancies with autologous HSP-peptides has been reported to result in induction of CTL response against autologous tumor cells. Response to the immunization and prolonged stabilization of disease have been discussed (19-24). These reports have sometimes suggested that HSP-peptide complexes have qualities of a tumor vaccine. However, the effectiveness of the immune response generated needs to be increased. Whereas immunization with HSP-peptide complexes derived from tumor cells has been reported to elicit CTL response and provides some partial protection, it fails to eliminate the tumors (10,13,17). These results indicate the need to improve to potency of chaperone protein-based agents.

There is a need for more effective tumor cell immunogenicity using HSP-peptide-based vaccines.

### SUMMARY OF THE INVENTION

The present invention relates to heat shock proteins (HSPs) and HSP complexes and aggregates thereof obtained from fused cells, for example heterohybrid cells. In some embodiments, the heterohybrid cells are generated from the fusion of at least one antigen presenting cell, such as but not limited to dendritic cells (DC) with at least one tumor cell.

The inventors have discovered that heat shock proteins (HSPs) and/or HSP complexes recovered from heterohybrid cells generated from the fusion of a dendritic cell (DC) and a tumor cell fusion (herein referred to as “heterohybrid DC-tumor fused cells”) can be used to induce a desired and/or specific immune response, such as for example, induction of antigen specific CD4⁺ and CD8⁺ T cells, stimulation of CD4⁺ and CD8⁺ T cells, stimulation of dendritic cells and/or induction of a CTL response etc. In particular, the inventors have discovered that HSPs and/or HSP complexes or aggregates thereof recovered from heterohybrid DC-tumor fused cells are significantly better than at preventing tumor growth and proliferation from a tumor cell challenge in an in vivo animal model of cancer as compared to HSPs and/or HSP complexes or aggregates thereof recovered from non-fused cells, for example from un-fused tumor cells.

Furthermore, the inventors have also discovered HSPs and/or HSP complexes or aggregates thereof recovered from heterohybrid DC-tumor cells are significantly better at inducing an immune response, such as induction of CD4⁺ and CD8⁺ T cells to tumor cells as compared to HSPs and/or HSP complexes or aggregates thereof recovered from non-fused cells, for example from un-fused tumor cells.

Accordingly, one aspect of the present invention relates to a method of inducing a CD4⁺ and/or CD8⁺ immune reaction to a population of tumor cells in a subject, comprising administering to the subject a chaperone or chaperone protein complex or aggregates thereof, recovered from a heterohybrid cell, wherein the heterohybrid cell is a fusion of at least one antigen presenting cell and at least one tumor cell.

In some embodiments, the CD4⁺ or CD8⁺ immune reaction is a specific CD4⁺ T cell response, and in some embodiments it is a specific CD8⁺ T cell response. In some embodiments, the CD4⁺ or CD8⁺ immune reaction is a CTL response. In some embodiments, the CD4⁺ or CD8⁺ immune reaction is a memory T-cell immune response, such as memory CD4⁺ T cells and/or memory CD8⁺ T cell response.

In some embodiments, heterohybrid cells useful in the methods as disclosed herein expresses at least one marker specific to the antigen presenting cell and at least one marker specific to the tumor cell. In some embodiments, examples of markers specific for antigen presenting cells which can be used can be selected from a group consisting of, for example but not limited to, CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II or homologues thereof.

In some embodiments, markers specific for tumor cells that can be used to identify a heterohybrid cell useful in the methods as disclosed herein can be a tumor-associated antigen or a marker selected from a group consisting of, for example but not limited to, prostate PSA, PSMA, Her-2/neu, HSP-70, EGF-R, MUC-1, Melan-A, MART1, p53, CEA, NYESO, TRP-1, TRP-2, MAGEs, BAGEs, TAG72 or GP-100 or homologues thereof.

In alternative embodiments, a heterohybrid cell useful in the methods as disclosed herein comprises the genetic material of more than one cell, for example the heterohybrid cell comprises at least two nuclei, or the heterohybrid cell is a tetraploid hybrid cell, which refers to a heterohybrid cell which has one larger nuclei as a result of the fusion of at the nuclei from at least two of the cells used in the fusion.

In some embodiments, a heterohybrid cell useful in the methods as disclosed herein a heterohybrid cell is a bikaryonic or trikaryonic heterohybrid cell.

In some embodiments, a tumor cell which can be used in the fusion to generate a heterohybrid cell useful in the methods as disclosed herein is allogenic and in some embodiments the tumor cell is autogenic. In some embodiments, the tumor cell can be any tumor cell known by persons of ordinary skill in the art, for example tumor cells expressing tumor-associated antigens, for example but not limited to PSA, PSMA, Her-2/neu, HSP-70, EGF-R, MUC-1, Melan-A, MART1, p53, CEA, NYESO, TRP-1, TRP-2, MAGEs, BAGEs, TAG72 or GP-100 or homologues thereof.

In some embodiments, the antigen presenting cell which can be used in the fusion to generate a heterohybrid cell useful in the methods as disclosed herein can be any antigen presenting cell known by persons of ordinary skill in the art. In some embodiments, the antigen presenting cell is a dendritic cell. In some embodiments, the antigen presenting cell is allogenic and in some embodiments the antigen presenting cell is autogenic. In some embodiments, the antigen presenting cell can be identified by cell-surface markers specific to the antigen presenting cell, for example but not limited to CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II or homologues thereof.

In some embodiments, the chaperone or chaperone protein complex or aggregates thereof recovered from heterohybrid cells which are useful in the method as disclosed herein is a heat shock protein, such as for example but not limited to, HSP70, HSP70-1a, HSP701b, HSP70-HOM, HSP70-4, HSP70-5, HSP70-9b, HSP75 or HSC70 or derivatives, isoforms or homologues thereof. In some embodiments, the heat shock protein is HSP70 or derivatives, isoforms or homologues thereof. In alternative embodiments, the heat shock protein can be, for example but not limited to gp96, BiP (Grp78), GrpE, HSP110, HSP90, HSP86, HSP84, HSP78, HSP75, HSP60, HSP40, HSP27, HSP20, α-crystallins, calreticulin or a derivatives, isoforms or homologues thereof.

In some embodiments, a tumor cell which can be used in the fusion to generate a heterohybrid cell useful in the methods as disclosed herein is a tumor cell from a human.

In some embodiments, the subject to which the chaperone or chaperone protein complex or aggregates thereof recovered from heterohybrid cells is administered to is a mammal, for example a human.

In some embodiments, administration of the chaperone or chaperone protein complex or aggregates thereof recovered from heterohybrid cells to a subject reduces immune tolerance in the subject to a population of tumor cells.

Another aspect of the present invention relates to a heat shock protein or heat shock protein complex or aggregates thereof recovered from a heterohybrid cell, wherein the heterohybrid cell is a fusion of at least one antigen presenting cell and at least one tumor cell.

In some embodiments, heterohybrid cells from which heat shock protein or heat shock protein complex or aggregates thereof can be recovered from expresses at least one marker specific to the antigen presenting cell and at least one marker specific to the tumor cell. In some embodiments, examples of markers specific for antigen presenting cells which can be used can be selected from a group consisting of, for example but not limited to, CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II or homologues thereof.

In some embodiments, markers specific for tumor cells that can be used to identify a heterohybrid cell from which heat shock protein or heat shock protein complex or aggregates thereof can be recovered from can be a tumor-associated antigen or a marker selected from a group consisting of, for example but not limited to, prostate PSA, PSMA, Her-2/neu, HSP-70, EGF-R, MUC-1, Melan-A, MART1, p53, CEA, NYESO, TRP-1, TRP-2, MAGEs, BAGEs, TAG72 or GP-100 or homologues thereof.

In alternative embodiments, a heterohybrid cell useful from which heat shock protein or heat shock protein complex or aggregates thereof can be recovered from comprises the genetic material of more than one cell, for example the heterohybrid cell comprises at least two nuclei, or the heterohybrid cell is a tetraploid hybrid cell, which refers to a heterohybrid cell which has one larger nuclei as a result of the fusion of at the nuclei from at least two of the cells used in the fusion.

In some embodiments, a heterohybrid cell from which heat shock protein or heat shock protein complex or aggregates thereof can be recovered from is a bikaryonic or trikaryonic heterohybrid cell.

In some embodiments, a tumor cell which can be used in the fusion to generate a heterohybrid cell from which heat shock protein or heat shock protein complex or aggregates thereof can be recovered from is allogenic and in some embodiments the tumor cell is autogenic. In some embodiments, the tumor cell can be any tumor cell known by persons of ordinary skill in the art, for example tumor cells expressing tumor-associated antigens, for example but not limited to PSA, PSMA, Her-2/neu, HSP-70, EGF-R, MUC-1, Melan-A, MARTI, p53, CEA, NYESO, TRP-1, TRP-2, MAGEs, BAGEs, TAG72 or GP-100 or homologues thereof.

In some embodiments, the antigen presenting cell which can be used in the fusion to generate a heterohybrid cell from which heat shock protein or heat shock protein complex or aggregates thereof can be recovered from can be any antigen presenting cell known by persons of ordinary skill in the art. In some embodiments, the antigen presenting cell is a dendritic cell. In some embodiments, the antigen presenting cell is allogenic and in some embodiments the antigen presenting cell is autogenic. In some embodiments, the antigen presenting cell can be identified by cell-surface markers specific to the antigen presenting cell, for example but not limited to CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II or homologues thereof.

In some embodiments, the heat shock protein or heat shock protein complex or aggregates thereof recovered from a heterohybrid cell can be heat shock protein or heat shock protein complex or aggregates such as for example, but are not limited to, HSP70, HSP70-1a, HSP701b, HSP70-HOM, HSP70-4, HSP70-5, HSP70-9b, HSP75 or HSC70 or derivatives, isoforms or homologues thereof. In some embodiments, the heat shock protein is HSP70 or derivatives, isoforms or homologues thereof. In alternative embodiments, the heat shock protein can be, for example but not limited to gp96, BiP (Grp78), GrpE, HSP110, HSP90, HSP86, HSP84, HSP78, HSP75, HSP60, HSP40, HSP27, HSP20, α-crystallins, calreticulin or a derivatives, isoforms or homologues thereof.

In some embodiments, a tumor cell which can be used in the fusion to generate a heterohybrid cell from which heat shock protein or heat shock protein complex or aggregates thereof can be recovered from is a tumor cell from a human.

Another aspect of the present invention relates to a method of inducing an antigen specific CD4 and CD8 T cell response in a subject comprising administering the heat shock protein or heat shock protein complex, or aggregates thereof, where the heat shock protein or heat shock protein complex or aggregates thereof are recovered from a heterohybrid cell, such as a heterohybrid cell generated from the fusion of at least one antigen presenting cell and at least one tumor cell.

In some embodiments, a subject which antigen specific CD4 and CD8 T cell response is induced is a human. In some embodiments, the subject has a disease or malady, for example the subject has cancer or a tumor.

Another aspect of the present invention relates to the use of a heat shock protein or heat shock protein complex recovered from a heterohybrid cell to modulate an immune response in a subject, wherein the heterohybrid cell is a fusion of at least one antigen presenting cell and at least one tumor cell.

A further aspect of the present invention relates to a composition for the treatment or prevention of cancer in a subject, the composition comprising a heat shock protein or heat shock protein complex or aggregates thereof recovered from a heterohybrid cell, where the heterohybrid cell is a fusion of at least one antigen presenting cell and at least one tumor cell.

### DESCRIPTION OF THE FIGURES

FIGS. 1A-1G show anti-tumor immunity induced by immunization with HSP70 complexes derived from DC-tumor fusion cells. FIG. 1A shows C57BL/6 wild-type mice (WT-mice, six mice per group) were immunized twice with 1.5 μg or 3 μg of HSP70-associated complexes from FC/MUC1 fusion cells (HSP.70-PC-F, white bar) or MC38/MUC1 tumor cells (HSP.70-PC-Tu, hatch bar) at the posterior flank near the base of tail on day 0 and 7, respectively. The mice injected with PBS (3/group, black bar) were used as control. On day 14, mice were challenged subcutaneously with 2×10⁵ MC38/MUC1 tumor cells. Tumor incidence (≧2 mm in diameter) was monitored up to 30 days. FIG. 1B shows splenocytes isolated on day 30 from WT-mice immunized with HSP.70-PC-F (white bar), HSP70-PC-Tu (hatch bar) or PBS (black bar) were incubated with MC38/MUC1 target cells at a E:T ratio of 50:1. FIG. 1C, MUC1-positive and negative targets were incubated with anti-MHC class I mAb (hatch bar) or IgG (gray bar) before addition of splenocytes isolated on from WT-mice immunized with HSP.70-PC-F. FIG. 1D shows MUC1.Tg mice (six mice per group) were vaccinated twice with 1.5 μg HSP70.PC-F (⊕) or HSP70.PC-Tu (□). The MUC1.Tg mice immunized with 2×10⁵ FC/MUC1 fusion cells (◯) or PBS (Δ) were used as control. One week after the second immunization, mice were challenged subcutaneously with 2×10⁵ MC38/MUC1 tumor cells. Tumor volume was measured by caliper at twice of week for 30 days. FIG. 1E shows CTL activity induced by immunization with HSP70 complexes derived from FC/MUC1. Splenocytes isolated from mice immunized with HSP70.PC-F (⊕), HSP70.PC-Tu (□), 2×10⁵ FC/MUC1 fusion cells (◯) or PBS (Δ) at 20 days after tumor challenge were incubated with MC38/MUC1 target cells at the indicated effector:target (E:T) ratios. FIG. 1F shows MUC1.Tg mice (six mice per group) were vaccinated twice with 1.5 μg HSP70-peptides from FC/MUC1 fusion cells (HSP70-peptide-F, ⊕) or MC38/MUC1 tumor cells (HSP70-peptide-Tu, □) purified by ADP-agarose column. The MUC1.Tg mice immunized with DC pulsed with purified HSP70-peptides (◯) or PBS (Δ) were used as control. Tumor volume was measured by caliper at twice of week up to 30 days. FIG. 1G shows CTL activity induced by immunization with purified HSP70-peptides derived from FC/MUC1. Splenocytes isolated from mice immunized with HSP70-peptide-F (⊕), HSP70-peptide-Tu (□), 2×10⁵ DC pulsed with HSP70-peptide-Tu (◯) or PBS (Δ) at 20 days after tumor challenge were incubated with MC38/MUC1 target cells at the indicated effector:target (E:T) ratios. (FIGS. 1A, D and F), the statistical significance of in vivo data was determined using χ² analysis (* and *** indicates P<0.05 and P<0.005, respectively. NS means no statistical significance). (FIGS. 1B, C, E and G), percentage of cytotoxicity was determined by standard ⁵¹Cr-release assay. In FIGS. 1B and C, the data was presented as mean±SD of three replicates.

FIGS. 2A-2F shows proliferation and activation of CD4 and CD8 T cells by HSP70-complexes derived from DC-tumor fusion cells. FIGS. 2A and B shows stimulation of naïve T cells in vitro. FIG. 2A shows whole LNC as compared to splenocytes, as shown in FIG. 2B, isolated from naïve MUC1.Tg mice and co-cultured with HSP70.PC-F (◯) or HSP70.PC-Tu (□) at indicated concentration. After 5 days, cells were pulsed with 1 μCi/well [³H] thymidine and harvested on filters. Radioactivity (mean±SD of triplicates) was measured by liquid scintillation counting. The measurement of cells cultured with medium alone was used as baseline. Results were obtained in three separate experiments. FIGS. 2C and 2D show stimulation of primed CD8 or CD4 T cells in vitro. T-LNC were isolated from vaccinated MUC1.Tg mice and then sorted to CD8 or CD4 T cells. FIG. 2C shows CD8 and FIG. 2D shows CD4 T cells from HSP70.PC-F immunized mice were co-cultured with DC and HSP70.PC-F (◯), and CD8 or CD4 T cells from HSP70.PC-Tu immunized mice were co-cultured with DC and HSP70.PC-Tu (□) at indicated concentration, respectively. After 5 days, the cultures were pulsed with 1 μCi per well [³H] thymidine and harvested on filters. Radioactivity was measured by liquid scintillation counting. The measurement of cells cultured with medium alone was used as baseline. Results were obtained in two separate experiments. FIGS. 2E and F shows in vivo activation of T cells by HSP70 complexes from DC-tumor fusion cells. LNC were freshly isolated from MUC1.Tg mice immunized twice with HSP70.PC-F, HSP70.PC-Tu or injection with PBS. The LNC were stained with mAbs against CD4, CD8, IFN-γ, CD69, CD44 and IL-15R and analyzed by flow cytometry.

FIGS. 3A-D shows the effect on maturation of DC by HSP70-complexes derived from DC-tumor fusion cells. FIGS. 3A and B shows the phenotype of DC stimulated by HSP70.PC-F or HSP70.PC-Tu. DC were generated from bone marrow cells of WT mice by antibody/complement treatment and cultured in the presence of GM-CSF medium. On third day, immature DC were selected by adherent method and further cultured overnight. Immature DC were then co-cultured with HSP70.PC-F or HSP70.PC-Tu (10 μg/ml) in the presence of GM-CSF medium. DC cultured in GM-CSF medium alone were used as control. FIG. 3A shows after 6 or 24 hour culture, the DC were collected, stained with indicated mAbs and analyzed by FACS. The expression of indicated molecules on DC co-cultured with HSP70.PC-F, HSP70.PC-Tu or medium alone was indicated by red, blue or black line, respectively. FIG. 3B shows quantification of DC positive for individual mAb co-cultured with HSP70.PC-F, HSP70.PC-Tu or medium is derived from three independent experiments. FIG. 3C shows immature DC were co-cultured with LPS, medium alone, HSP70.PC-F, boiled HSP70.PC-F, HSP70.PC-Tu, HSP70-PC from tumor-tumor fusions (HSP70.PC-Tu+PEG) or mixture of DC and tumor cells (HSP70.PC-Tu+DC). After 24 hour culture, the DC were collected and analyzed for the expression of CD86. FIG. 3D shows Inguinal LN collected from mice immunized with HSP70.PC-F, HSP70.PC-Tu or PBS were frozen and cryosectioned. The sections were stained with anti-CD86 (red color) and examined under microscopy (Magnification, x100). (B and C), the statistical significance of DC maturation was determined using χ² analysis (* and *** indicates P<0.05 and 0.005, respectively).

FIGS. 4A-4E shows a comparison of DC phenotype from WT and MyD88 KO mice stimulated by HSP70.PC-F. As shown in FIGS. 4A and 4B, DC were generated from wild-type (WT-DC, red line) or MyD88KO (MyD88-DC, green line) mice using the same method as described in FIG. 3 and co-cultured with HSP70.PC-F in the presence of GM-CSF medium for 6 (FIG. 4A) or 24 hours (FIG. 4B). WT-DC or MyD88-DC cultured in medium containing GM-CSF (black line) were used as controls. The harvested DC were stained with indicated mAbs and analyzed by FACS. FIG. 4C shows quantification of DC positive for individual mAb is derived from two independent experiments. The statistical significance of DC maturation was determined using χ² analysis (*** indicates P<0.005). FIG. 4D shows T cell proliferation. LNC were isolated from WT, MUC1.Tg or MyD88 KO mice immunized with either HSP70.PC-F (◯) or HSP70.PC-Tu (Δ). After 5 day culture with 5 μg/mlHSP70.PC-F or HSP70.PC-Tu, respectively, the cells were pulsed with 1 μCi per well [³H] thymidine and harvested on filters. Radioactivity was measured by liquid scintillation counting. FIG. 4E shows CTL assay where splenocytes were isolated from WT, MUC1.Tg or MyD88 KO mice immunized with either HSP70.PC-F or HSP70.PC-Tu as effector cells. MUC1-positive and negative targets were labeled with ⁵¹Cr and then mixed with splenocytes isolated from mice immunized with HSP70.PC-Tu (blue bar) or HSP70.PC-F (orange bar) at a E:T ratio of 60:1. Percentage of cytotoxicity was determined by standard ⁵¹Cr-release assay.

FIGS. 5A-D show chaperoning of MUC1 by HSP70 derived from FC/MUC1 fusion cells. FIG. 5A shows immunoblotting analysis of HSP SDS contents in cell lysates from DC, MC38/MUC1 tumor cells, and FC/MUC1 fusion cells treated with or without HS at 43° C. for 1 hour and 37° C. for 6 hours. Lysates were analyzed by SDS-PAGE followed by immunoblotting with antibodies against HSP-25, -70, -90 or -110. Equal loading of the protein was determined by SDS-PAGE. FIG. 5B shows the association of HSP70 with MUC1. Lysates from HS-treated FC/MUC1 fusion cells, MC38/MUC1 tumor cells or DC alone were immunoprecipitated with anti-HSP70 or anti-MHC class II antibodies followed by immunoblotting with anti-MUC1 mAb DF3. The relative density of MUC1 in lane 4 and 6 was determined by densitometric analysis of X-ray films in the chemiluminescence-based protein detection. FIG. 5C shows detection of MUC1 peptide from GST-HSP70 complexes by anti-MUC1 peptide antibody. GST-HSP70-complexes were pulled down from GST-HSP70 transfected MC38/MUC1 tumor cells (Δ), DC fused with GST-HSP70 transfected MC38/MUC1 cells (FC/MUC1/GST-HSP70, ◯) or vector-transfected tumor cells (□) and used to coat the ELISA plate at indicated concentrations for ON at 4° C. After washing 5 times, the anti-MUC1 peptide antibodies BCP8 (left panel) or BCP9 (right panel) were added into the plate for 2 h at RT. Following washing and blocking, the second antibody HRP-anti mouse IgG at 1:5000 dilution was added and the color was developed by o-phenylenediamine tetrahydrochloride buffer with H₂O₂. The absorbance was measured by an autoreader at 492 nm. FIG. 5D shows a comparison of MUC1 peptide in HSP70-PC obtained by GST pull-down or ADP affinity chromatography. The GST-HSP70-complexes pulled down from fusion cells (◯) or tumor cells (Δ) (left panel), and HSP70-PC purified by ADP affinity Chromatography from fusion cells (◯) or tumor cells (Δ) (right panel) was assayed with BCP8 mAb using the same method in FIG. 5C. Statistical significance as used in FIGS. 5C and 5D was determined using Student's t test (*** indicates P<0.005).

FIGS. 6A-6C show increased association of HSP70 from fusion cells with HSP90. FIG. 6A shows detection of HSP70-PC associated with other HSPs using Weston Blot. DC mixed with MC38/MUC1 (lane 1), MC38/MUC1 tumor cells (lane 2) or FC/MUC1 fusion cells (lane 3) were lysed by lysis buffer. The lysates were subjected to immunoprecipitation with anti-HSP70 mAb (Ab46). The precipitates were analyzed by immunoblotting with indicated antibodies in 10% SDS gel. FIG. 6B shows lysates from MC38/MUC1 tumor cells (lane 1), FC/MUC1 (lane 2), FC/MUC1 treated with geldanamycin (1 μg/ml) (lane 3) were immunoprecipitated with anti-HSP70 antibody (Ab 46). The precipitates were analyzed by immunoblotting with anti-HSP90 antibody in 6% SDS gel. Line 4 is lysates from MC38/MUC1 tumor cells. FIG. 6C shows LNC and splenocytes were isolated from naïve mice and co-cultured with HSP70.PC-Tu, or HSP70.PC-F treated with or without geldanamycin (GA) and then measured for T cell proliferation with standard [³H] thymidine incorporation assay. The data are representative of three separate experiments.

FIG. 7 shows an assessment of HSP70-PC derived from human DC fused to tumor cells from a breast cancer cell line. FIG. 7A shows DC generated from a PBMC of a healthy donor were fused to BT20 (FC/BT20). DC and FC/BT20 were stained with antibodies against MHC class II, MUC1 and/or MUC1 peptide DTRPAPGST (BCP-8) or APDTRPAPG (BC-2) and then analyzed by flow cytometry. After 5 day culture, the BT20 tumor and FC/BT20 fusion cells were lysed in the lysis buffer and immunoprecipitated by anti-HSP70 antibody (AB-46). FIG. 7B shows stimulation of naïve PBMC cells in vitro. The PBMC from the same donor from whom DC were generated were cocultured with HSP70.PC-FC/BT20 (▪) or HSP70.PC-BT20 () at indicated concentration for 5 days and the T cell proliferation was measured using the standard [³H]-thymidine incorporation. The measurements were presented as mean±SD of triplicates using cells cultured with medium alone as baseline. FIG. 7C shows activation of T cells by HSP70.FC-BT20. PBMC were cocultured without or with 5 μg/ml HSP70.PC-FC/BT20 or HSP70.PC-BT20 for 5 days, and then collected, purified and stained with triple antibodies (IFN-γ-FITC, CD69-PE and CD4 or CD8-Cy from BD Pharmigen) and analyzed by flow cytometry.

FIG. 8 shows enhanced CTL activity induced by HSP70-PC extracted from DC-tumor fusion cells. DC were generated from PBMC of a healthy donor and fused to MCF7, SKBR3 and BT20 breast cancer cells. HSP70-PC were extracted from FC/MCF7, FC/SKBR3 and FC/BT20, respectively, using anti-HSP70 antibody (AB-46). FIG. 8A shows enhanced CTL activity against breast cancer cells. PBMC positive for HLA-A*02 and HLA-A*11 co-cultured with HSP70.PC-FC/MCF7, HSP70.PC-FC/SKBR3 or HSP70.PC-FC/BT20 for 5 days were incubated with ⁵¹Cr-labeled MCF7, SKBR3 and BT20 breast cancer cells, respectively, at E:T ratio of 50:1 and assayed for ⁵¹Cr release. PBMC cultured with HSP70.PC extracted from MCF7, SKBR3 or BT20 breast cancer cells were used as control at the same E:T ratio. FIG. 8B shows PBMC positive for HLA-A*02 and HLA-A*11 were co-cultured with HSP70.PC-FC/MCF7, HSP70.PC-FC/SKBR3 or HSP70.PC-FC/BT20. Five days late, T cells were purified by passage through nylon wool and cultured with ⁵¹Cr-labeled MCF7, SKBR3 and BT20 breast cancer cells and MC pulsed with MUC1 or MC alone at 50:1 E:T ratio. CTL activity against indicated targets were determined by ⁵¹Cr release assay.

### DETAILED DESCRIPTION

The present invention relates to heat shock proteins (HSPs) and HSP complexes and aggregates thereof obtained from fused cells, for example heterohybrid cells generated from the fusion of dendritic cells (DC) with tumor cells. The inventors have discovered that heat shock proteins (HSPs) and/or HSP complexes derived from heterohybrid cells of dendritic cell (DC) and tumor cells fusion (herein referred to as “heterohybrid DC-tumor fused cells”) can be used to induce a desired and/or specific immune response, such as for example, induction of antigen specific CD4⁺ and CD8⁺ T cells, stimulation of CD4⁺ and CD8⁺ T cells, stimulation of dendritic cells and/or induction of a CTL response etc.

In some embodiments, HSPs and/or HSP complexes derived from heterohybrid DC-tumor fused cells can be extracted and used to induce or elicit an immune response against a tumor cell or tumor. In such embodiments, the present invention provides methods to reverse or reduce the immune tolerance of the tumor cell or cancer cell using the HSPs and/or HSP complex as disclosed herein. Accordingly, one embodiment of the present invention provides methods for reversing immune tolerance of tumors and tumor cells.

In one embodiment, the method of the present invention comprises the steps of: (i) fusing at least one dendritic cell (DC) with at least one tumor cell or a population of tumor cells (for example, tumor cells one is wishing to break immune tolerance to), and (ii) extracting a HSP and/or HSP complexes from the fused cell or a population of fused cells, for example extracting HSP70 complexes, which comprise a HSP70 protein or fragment thereof and protein fragment complexes, such as protein fragments of cellular and/or cytosolic proteins of the heterohybrid cell), and (iii) administering the HSPs and/or HSP complexed to a subject to induce an immune response and to break immune tolerance of the tumor cell.

In one embodiment, the present invention relates to the use of chaperone proteins and chaperone protein complexes or aggregates thereof obtained from a sample of heterohybrid cells comprising fused cells, where the fused cells are generated from the fusion of two or more cells from different origins.

The inventors have discovered that HSPs and HSP complexes obtained from heterohybrid cells, such as heterohybrid DC-tumor fused cells have a significantly better repertoire of antigenic properties as compared to HSPs and HSP complexes obtained from non-fused cells. Accordingly, the HSPs and HSP complexes obtained from heterohybrid cells, such as heterohybrid DC-tumor fused as disclosed herein can be used to accomplish at least one of the following: to prevent or reduce T cell tolerance to defined tumor antigens, kill or reduce proliferation of tumor cells, prevent tumor growth, initiate host DC maturation, lead to expression of co-stimulatory molecules required for T cell activation, active effectors and/or memory T cells.

In one embodiment, the present invention provides a method to prepare HSP and/or HSP protein complexed or aggregates thereof from heterohybrid cells, such as heterohybrid DC-tumor fused cells.

The inventors have discovered that HSPs and HSP complexes as disclosed herein have several advantages over previously described chaperone protein and chaperone protein complexes, such as HSP proteins and HSP complexes derived from non-fused cells, in that HSP and HSP complexes isolated from heterohybrid cells induce a greater CTL response and provide better protection as compared to chaperone proteins or chaperone protein complexes (for example HSP and HSP complexes obtained from non-fused cell populations) against subsequent challenged with cells derived from the same type of tumor.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “heterohybrid cell” as used herein refers to a cell originated from the fusion of a one type of cell (Type I cell) and one or more different types of cells (Type II cell). The heterohybrid cells involved in the invention comprise mainly of bikaryons but could include trikaryons and multiples or bikaryons and trikaryons. Heterohybrid cells are generated from the fusion of a minimum of two different types of cells (Type I and Type II) and any number of different types of cells. Homohybrid cells refer to cells generated from the fusion of two or more cells of the same type, for instance from the fusion of at least two Type I cells or the fusion of at least two Type II cells. In some embodiments, one or more Type I cells can be fused with more than one Type II cells to generate a heterohybrid cell useful in the methods and compositions of the present invention.

In some embodiments, a heterohybrid cell can be generated from the fusion of at least one Type I cell with at least one Type II cells, which can be further fused with either at least one Type I and/or at least one Type II cell. In some embodiments, a heterohybrid cell as disclose herein can be fused with at least one bikaryonic and/or trikaryonin heterohybrid or homohybrid cell comprising Type I and/or type II cells.

The term “fusion” or “fused” as used herein refers to the joining of one cell with another, wherein the cytoplasm of each cell is integrated with the other cell and the exchange cytoplasmic contents and subcellular compartments is possible. As an example, the present invention may take advantage of fused cells produced according to the methods described in U.S. Patent Application No. 2006/0068495, which is incorporated herein in its entirety by reference.

As used herein, the “type of cell” refers to a cell having a defined function and characteristic, for example, but not limited to antigen presenting cells, lymphocyte or a tumor cell originating from any tissue or any cellular preparation.

The term “cell population enriched in the heterohybrid cells” as used herein refers to a cell population which has a higher ratio of the heterohybrid cells as compared to non-fused cells or homohybrid cells, where the heterohybrid cells are fused cells from one or more different types of cells. For example, a population enriched for heterohybrid cells means that the cell population, in this case heterohybrid, is the predominant species, but the cell population may also contain non-fused cells and homohybrid cells.

The term “chaperone protein-protein complex’ or “chaperone protein complex” as used herein refers to a complex of any or one or more chaperone proteins to form a multi-chaperone complex and any or a multitude of other proteins. The other proteins or chaperone proteins can be denatured or non-denatured. The other proteins or chaperone proteins associated with the chaperone protein in the chaperone protein complex can be a naturally occurring protein, a protein produced by genetically engineered cell, or a synthetic protein. The proteins can become associated with chaperone proteins inside a cell, i.e. endogenously or outside the cell i.e. exogenously. The protein can be non-covalently associated with a chaperone protein or covalently linked to the chaperone.

The term “chaperone protein complex” and “HSP complexes” are used interchangeably herein and refers to a compositions of chaperone proteins, chaperone protein complexes, or aggregates thereof, wherein said chaperone proteins, chaperone protein complexes, or aggregates thereof, are enriched from the biological sample of heterohybrid cells.

By “naturally occurring protein” as used herein refers to chaperone proteins which form a multi-chaperone complex and/or accessory proteins and/or co-chaperone proteins. Other proteins may be denatured or non-denatured proteins, comprising entire proteins or parts of proteins, such as peptides. Proteins may be mutated proteins or non-mutated proteins.

The term “synthetic protein” as used herein refers to a protein produced by an artificial means as known to persons of ordinary skill in the art, for example, but not limiting to, synthetic peptides, recombinant proteins, fusion proteins or chimeric proteins. The synthetic protein can be from either prokaryotic or eukaryotic organisms.

The term “aggregates” as used in the context of HSP complexes or chaperone proteins or chaperone protein complexes refers to a complex of one or more different HSPs or HSP complexes or chaperone proteins and/or chaperone protein complexes. For example, but not as a limitation, aggregates can comprise any or all of the following, but are not limited to: gp96, HSP110 and HSP110 isoforms, HSP90 and HSP90 isoforms HSP86 and HSP84, HSP70 and HSP70 isoforms HSP70, HSP78, HSP75, and HSC70, HSP60, HSP40, the small heat shock protein family (sHsp); HSP27 and HSP20 and other sHSPs, and calreticulin (CRT) and complexes thereof.

The term “accessory protein” or “co-chaperone” are used interchangeably herein refers to any molecule or protein that facilitates the activity of the chaperone protein. As an example, but not as a limitation, the accessory proteins or co-chaperones of the HSP70 family comprise of, but are not limited to, HDJ1, GrpE, BAG-1, HSPBP1, Hop, CHIP and HSP90.

The term “electrical field” as used herein refers to an electrical field applied by using any device and reaction conditions known by a person of ordinary skill in the art in applying electric fields to cells as to modify them while keeping them alive. The electrical field is applied as a succession of electrical impulses.

The terms “patient”, “subject” and “individual” are used interchangeably herein, and refer to an animal, particularly a human, to whom treatment including prophylaxic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term “antibody” as used herein includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarily determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The antibodies can be polyclonal or monoclonal and can be isolated from any suitable biological source, e.g., murine, rat, sheep and canine. Additional sources are identified infra. The term “antibody” is further intended to encompass digestion fragments, specified portions, derivatives and variants thereof, including antibody mimetics or comprising portions of antibodies that mimic the; structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)² fragment, a bivalent fragment comprising two Fab fragments linked by a disulfide bridge at the hinge region; a Ed fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarily determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “cancer” or “tumor” or “malignancy” are used interchangeably herein, refers to any tissue mass or tissue type or cell type that is undergoing uncontrolled proliferation, or abnormal growth, and refers to a cellular proliferative disease in a subject, for example a human or animal subject. Cancer cells can spread locally or through the bloodstream and lymphatic system to other parts of the body. The term “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer.

The term “tumor cell” as used herein refers to any cell undergoing uncontrolled proliferation or abnormal growth or expresses specific tumor-associated antigens.

The term “antigen presenting cell” as used herein broadly refers to a cell that carries on its surface receptors that bind antigens via MCH Class I or Class II molecules and presents the antigen in this context to T-cells. Antigen presenting cells includes, for example macrophages, endothelium, dendritic cells and Langerhans cells of the skin.

The term “disease” or “disorder” are used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection. A disease and disorder, includes but is not limited to any condition manifested as one or more physical and/or psychological symptoms for which treatment is desirable, and includes previously and newly identified diseases and other disorders.

The term “comprising” means “including principally, but not necessary solely”. Furthermore, variation of the word “comprising”, such as “comprise” and “comprises”, have correspondingly varied meanings.

The articles “a” and “an” are used herein to refer to one or to more than one (i.e., to at least one) of the grammatical object of the article. By way of example, “an element” means one element or more than one element.

### Heterohybrid Cells

One aspect of the present invention relates to obtaining HSPs and HSP complexes from heterohybrid cells, for example from DC-tumor heterohybrid fused cells. As defined herein, a heterohybrid cell is a fused cell (sometimes referred to in the art as a hybrid cell) as a result of the fusion of a one type of cell (herein referred to as a Type I cell) with one or more different types of cells (herein referred to a Type II cell).

In some embodiments, the HSPs and HSP complexes useful in the methods ad disclosed herein are obtained from bikaryons heterohybrid cells, for example a heterohybrid DC-tumor fused cell or alternatively from a trikaryon heterohybrid cells (such as a DC-DC-tumor fused cell or DC-tumor-tumor fused cell) and multiples or bikaryons and trikaryons heterohybrid cells. In some embodiments, HSPs and HSP complexes useful in the methods as disclosed herein are obtained from fused cells generated from the fusion of one or more Type I cells with at one or more Type II, or from a heterohybrid cell that has been further fused with either at least one Type I and/or at least one Type II cell. In some embodiments, HSPs and HSP complexes can be obtained from heterohybrid cells that have be fused with at least one bikaryonic and/or trikaryonin heterohybrid or homohybrid cell comprising Type I and/or type II cells.

In some embodiments, Type I and/or Type II cells useful in the fusion to generate heterohybrid cells as disclosed herein can be modified by persons of ordinary skill in the art, for example the cells can be genetically modified to include heterologous nucleic acids. For example type I and/or type II cells, or heterohybrid cells can be transfected and/or transformed with one or more genes, such as, but are not limited to, genes encoding chaperones, such as heat shock proteins (HSPs), chaperone accessory proteins, co-chaperones, antigens of an infectious agent (such as a bacterial toxin agent, as a non-limiting example, an anthrax toxin agent or fragment thereof), tumor antigens, or fragments thereof. For example, cells can be modified to have the genes of heat shock proteins or nucleic acids encoding fragments of HSPs introduced, such as the genes or fragments thereof of HSPs such as, but not limited to, HSP70-1a, HSP70-1b, HSP70-HOM, HSP70-4, HSP70-5, HSP70-8, HSP70-9b, HSP75 and HSC70 or genes encoding their co-chaperones or co-factors or fragments thereof.

Any method to modify a cell can be used in the methods of the present invention, and such methods are commonly known by persons of ordinary skill in the art. For example, but not as a limitation, by an infectious agent such as a virus or viral vector, by naked DNA, use of RNAi technologies or use of cells from transgenic or genetically engineered animals or by other known means. Modification of Type I and/or Type II cells can be done prior to cell fusion, during cell fusion or once the cells are heterohybrid fused cells.

When cellular heterohybrids are intended to be used for the treatment of a patient, cells before fusion may be either autologous (auto) or allogenic (allo) cells relatively to the patient. Preferably, the cells are autologous. Autologous cells may come from sample taking such as blood taking, or from tissue biopsy. Allogenic cells may come from cell lineages, such as characterized lineages accessible to libraries such as the American Tissue Culture Collection (ATCC) (U.S.A). Therefore, different types of heterohybrids may be generated and characterized relatively to the patient according to their auto/auto, auto/allo, allo/auto or allo/allo origin or any number of combinations.

In one embodiment of the present invention, the types of cells used in the fusion are Type I and Type II cells.

In another embodiment, the Type I and/or Type II cells used for fusion can be mammalian cells or non-mammalian cells.

In another embodiment, Type I and/or Type II cells used for fusion can be mammalian cells. Type I and Type II cells can be from humans or from animals in general, for example, mammals, non-human animals such as farm animals comprising, but not limited to: cattle, horses; goats; sheep; pigs; donkeys; etc. household pets including, but not limited to: cats; dogs; rodents comprising but not limited to: rabbits, mice; hamsters; etc; birds and poultry and other livestock and fowl.

In another embodiment, any continuous source or primary cell line of Type I and Type II cells, known to the person skilled in the art, can serve as the source of cells for fusion to generate heterohybrids.

### Type I Cells

The term “Type I cell” as used herein refers to any cell that functions an antigen presenting cell. A Type I cell can also encompass any cell that assists in the function of an antigen presenting cell (APC).

In one embodiment of the invention, one of the types of Type I cell used in the fusion to generate a heterohybrid cell is an antigen presenting cell (APC). As a non-limiting example, APCs may be dendritic cells or macrophages.

Antigen presenting cells express high levels of MCH Class I and class II as well as co-stimulatory molecules. The fusion of an APC (Type I cell) with a second type (Type II cell) of cell expressing some antigens on its surface allows the preparation of a cellular heterohybrid expressing both co-stimulatory molecules from the APC and surface antigens from said second type of cell, said heterohybrid cell having higher immunogenicity than the second type of cell (Type II cell) alone.

In some embodiments, an antigen presenting cells as used herein is a dendritic cell (DC), macrophages or B lympocytes. In some embodiments, a DC can be an immature or mature DC. In some embodiments, the DC is a mature DC, the higher level of expression of surface markers on mature DCs rendering them preferable.

Without being bound by theory, one of the most efficient antigen-presenting cells (APCs) are mature, immunologically competent debdritic cells (DCs). DCs are capable of evolving from immature, antigen-capturing cells to mature, antigen-presenting, T cell-priming cells; converting antigens into immunogens and expressing molecules such as cytokines, chemokines, costimulatory molecules and proteases to initiate an immune response. The types of T cell-mediated immune responses (tolerance vs. immunity, Th1 vs. Th2) induced can vary, however, depending on the specific DC lineage (myeloid DC1s or lymphoid DC2s) and maturation stage in addition to the activation signals received from the surrounding microenvironment.

DCs are derived from hematopoietic stem cells in the bone marrow and are widely distributed as immature cells within all tissues. Immature DCs are recruited to sites of inflammation in peripheral tissues following pathogen invasion. Internalization of foreign antigens can subsequently trigger their maturation and migration from peripheral tissues to lymphoid organs. Immature DCs may express the chemokine receptors CCR1, CCR2, CCR5, CCR6 and CXCR1, and thus can be chemoattracted to areas of inflammation primarily by MIP-3a/CCL20, but also in response to RANTES/CCL5 and MIP-1a/CCL3. Following antigen acquisition and processing, DCs migrate to T cell-rich areas within lymphoid organs via blood or lymph, simultaneously undergoing maturation and modulation of chemokine and chemokine receptor expression profiles, with a change in expression levels of CCR6 and CCR7 occurs DC maturation.

The most prevalent antigen receptors expressed by DCs include members of the C-type lectin family, such as DEC-205, DCIR, dectin-2, CLEC-1. The macrophage mannose receptor (MR) and Fc receptors for immunoglobulins, FcγR and FcγRI, are also involved in antigen handling by immature DCs. DCIR, MR, FcγR are all down-regulated upon DC maturation, further emphasizing their specific roles in antigen uptake in immature DCs. A variety of additional cell surface receptors are expressed by DCs, for example, but not limited to, Down-Regulated by Activation (DORA), Immunoglobulin-like transcript 3 (ILT3); hsp receptors expressed at the cell surface of immature DC; Toll-like receptors (TLRs) expressed on immature DCs (e.g. TLR3) and CD47, a thrombospondin receptor. Intracellular proteins are also involved in antigen uptake and processing by DCs. Following antigen processing, antigenic peptides may then be presented via MHC molecules on the DC surface to CD4+, CD8+ or memory T cells. DCs are capable of processing both exogenous and endogenous antigens and present peptide in the context of either MHC class I or II molecules. As DCs mature, they transport peptide-loaded MHC class II complexes to the cell surface, which coincides with increased expression of costimulatory molecules, such as B7-1/CD80 and B7-2/CD86, which serve to amplify T cell receptor (TCR) signaling and promote T cell activation. DC-LAMP, is specifically expressed in the lysosomal MHC II compartment and is up-regulated following CD40 ligation on DCs. Immature DCs may also express empty MHC II and HLA-DM at their cell surface which later disappear upon maturation. DCs present antigenic peptides complexed with MHC class 1 molecules to CD8-expressing T cells in order to generate cytotoxic cells. In the event that DCs are themselves infected with a virus, proteasomes can simply degrade the viral proteins into peptides and transport them from the cytosol to the endoplasmic reticulum. The transporter associated with protein processing (TAP-1 and -2) is a dedicated peptide transporter that facilitates the transfer of cytosolic peptides to the endoplasmic reticulum where they can then bind to MHC class I molecules. DCs thus “cross-prime” T cells.

A variety of cell surface receptors expressed by immature DCs function in antigen uptake and also present antigen via the MHC I pathway. DCs use av-containing integrins and CD36, Hsp/antigen complexes can bind CD91 on DCs and be delivered via TAP-1 and -2 to the endoplasmic reticulum for MHC I-antigen presentation. FcγR-mediated internalization of immune complexes also results in presentation of exogenous antigen with MHC I.

Following antigen exposure and activation, DCs migrate into T cell areas of lymphoid organs, and mature DCs lose responsiveness to MIP-3a/CCL20, RANTES/CCL5 and MIP-1a/CCL3 and become particularly sensitive to MIP-3β/CCL19, and lose cell surface expression of CCR1, CCR5 and CCR6, down-regulate CXCR1 and up-regulate expression of CXCR4 and CCR7. Maturation of DCs also induces the production of MDC/CCL22, TARC/CCL17 and PARC/CCL18. DC production of the chemokine CXCL16, in T cell-rich areas of lymphoid organs, also functions in promoting interaction between DCs and cytotoxic T cells.

Cell surface receptors expressed by DC cells also mediate physical contact between DCs and T cells. DC-specific intercellular adhesion molecule (ICAM)-3 grabbing non-integrin (DC-SIGN), is a DC-specific ligand for ICAM-3 expressed on naïve T cells. Dectin-1, a DC-specific type II C-type lectin binds to receptors on T cells. Adhesion receptors such as lymphocyte function-associated antigen (LFA)-1, ICAM-1, LFA-3 and CD44 are also expressed on mature DCs and promote adhesion to T cells. The soluble cytokine profile secreted by DCs varies with the different stages of DC development and maturation thus influencing the different effector functions characteristic of immature vs. mature DCs. Mature DCs can express a wide variety of cytokines (not necessarily simultaneously) including IL-12, IL-1α, IL-1β, IL-15, IL-18, IFN-α, IFN-β, IFN-γ, IL-4, IL-10, IL-6, IL-17, IL-16, TNF-α, and MIF, and the distinct cytokine patterns released by mature DCs determine their Th1/Th2 polarizing capacities. Antigens that prime DCs to secrete IL-12 will typically induce Th1 differentiation, whereas antigens that do not elicit or, on the contrary, inhibit IL-12 production (e.g. IL-10, prostaglandin E2, cholera toxin) will promote Th2 differentiation.

Receptor-ligand interaction between TNF superfamily receptors (TNFSFRs) and their corresponding ligands influences both DC maturation and T cell priming. Fas engagement on immature DCs, for example, induces both their maturation and release of IL-1β and IFN-γ. Ligation of CD40 promotes an up-regulation of the costimulatory molecules B7-1/CD80 and B7-2/CD86, IL-12 secretion and release of chemokines (e.g. IL-8, MIP-1a, MIP-1β). DCs also up-regulate OX40L, in response to CD40 ligation, where OX40L binding to OX40 enhances cytokine production by DCs (e.g. TNF-α, IL-12, IL-1β and IL-6) and also induces expression of costimulatory molecules.

Mature DCs have a finite life expectancy, however, ligation of TRANCE R/RANK on their cell surfaces leads to increased survival. The activity of various cytokines and chemokines secreted by mature DCs and T cells can be modulated by DC-associated proteases and protease inhibitors. A novel serpin (serine protease inhibitor) secreted by DCs, PI-11, may function in promotion of the immune response by protecting cytokines from degradation. Two different disintegrin proteases, decysin and MADDAM/ADAM19 (metalloprotease and disintegrin dendritic antigen marker/a metalloprotease and disintegrin 19), have been identified as markers for DC differentiation. Disintegrins may potentially influence proteolysis, adhesion, fusion and intracellular signaling. TACE/ADAM17, for example, has been shown to regulate the activity of TNF-α by cleaving the membrane-bound form to produce soluble TNF-α. Decysin and MADDAM/ADAM19 may also play similar roles in modulating the cytokine microenvironment of DCs and T cells.

Accordingly, mature or immature DC cells will express at least one of the following specific cell surface marker at some time point; CCR1, CCR2, CCR5, CCR6, CXCR1, CCR7, TRANCE R/RANK, OX40L, LFA-1, ICAM-1, LFA-3, CD44, ICAM-3, DC-SIGN, CXCR4, CCR7, MDC/CCL22, TARC/CCL17, PARC/CCL18, CD91, DC-LAMP, B7-1/CD80, B7-2/CD86, CD47, LR3, DORA, ILT-3; FcγR, FcγRI, DEC-205, DCIR, dectin-2, CLEC-1, MR, and are useful in the methods as disclosed herein to identify DC cells and heterohybrid cells generated from the fusion of a DC with another cell.

The ability of DCs to regulate immunity is dependent on DC maturation. A variety of factors can induce maturation following antigen uptake and processing within DCs, including: whole bacteria or bacterial-derived antigens (e.g. lipopolysaccharide, LPS), inflammatory cytokines, ligation of select cell surface receptors (e.g. CD40) and viral products (e.g. double-stranded RNA). During their conversion from immature to mature cells, DCs undergo a number of phenotypical and functional changes. The process of DC maturation, in general, involves a redistribution of major histocompatibility complex (MHC) molecules from intracellular endocytic compartments to the DC surface, down-regulation of antigen internalization, an increase in the surface expression of costimulatory molecules, morphological changes (e.g. formation of dendrites), cytoskeleton re-organization, secretion of chemokines, cytokines and proteases, and surface expression of adhesion molecules and chemokine receptors.

In another embodiment of the invention, the antigen presenting cell is a monocyte-derived antigen presenting cell (MD-APC). MD-APCs are obtainable by the culture of monocytes in presence of specific differentiation factors. As an example, the present invention may take advantage of MD-APCs produced according to the methods described in WO 94/26875, WO 97/44441, and U.S. Pat. No. 5,662,899 or U.S. Pat. No. 5,804,442. Mature dendritic cells may be obtained according to known methods such as Bacaccio et al (2002) (Bacaccio et al, (2002) J Immunother, 25(1); 88-86).

MD-APCs may be autologous or allogenetic to a patient to which heterohybrids are intended to be administered. If allogenic MD-APCs are to be used, they may be selected from the same haplotype as the one of the patient for at least the main determinants of class I subtype.

### Type II Cells

The term “Type II cell” as used herein refers to any type of cell comprising relevant antigens. This comprises, but it not limited to tumor cells, cancerous cells, infected cells, cells containing misfolded proteins that contribute to a disease, genetically modified cells, pathogenic cells etc.

In another embodiment of the invention, the second type of cell (Type II cell) used in the generation of heterohybrid cells are tumor or cancer cells, either alive or treated so as to be killed or detoxified.

Tumor cells may originate from a tumor lineage, possibly expressing known antigens, and available for example at ATCC or as lines derived from a patient tumor. Cells may also come from a fragment of tumor excised from a patient to be treated. Tumor cells may be killed before fusion, by using any method known by person skilled in the art, such as γ-irradiation or applying an electrical field.

In another embodiment, one or more of the Type II cells used in fusion can be cancer cells or precancerous cells isolated from each individual patient (e.g. preferably prepared from infectious tissues or tumor biopsies of the patient). Any tissues, or cells isolated from the cancer, including cancer that has metastasized to multiple sites, can be used for fusion with one or more cells to generate heterohybrid cells. For example, but not limited to, solid tumor tissue (e.g. primary tissue from a biopsy) can be used, leukemic cells circulating in the blood, lymph or other bodily fluids can also be used.

In some embodiments, tumor cells to be used in the generation of heterohybrid cells can be tumor cells expressing tumor antigens or tumor associated antigens. Such tumor antigens or tumor associated antigens are, for example but are not limited to, oncogenes, such as . . . , prostate PSA or PSMA, Her-2/neu, HSP-70, HER2/Her-2, BRAC1, BRAC2, Rb, p53, EGF-R, MUC-1, Melan-A, MART1, p53, CEA, NYESO, TRP-1, TRP-2, MAGEs, BAGEs, TAG72, GP-100; sialyl-Lewis oligosaccharides, selectine ligands, such as O-glycanes or gangliosides, which are glycanic antigens associated to cancer, in particular LH or MUC-1 antigens.

In some embodiments, the tumor cells express specific cell-surface markers on their surface, for example but not limited to PSA or PSMA, Her-2/neu, HSP-70, HER2/Her-2, BRAC1, BRAC2, Rb, p53, EGF-R, MUC-1, Melan-A, MART1, p53, CEA, NYESO, TRP-1, TRP-2, MAGEs, BAGEs, TAG72, GP-100; sialyl-Lewis oligosaccharides, selectine ligands, such as O-glycanes or gangliosides.

In another embodiment of the invention, one or more of the Type II cells used in fusion can be tumor cells of mescenchymal in origin (sarcomas) i.e. cancers of the following types: fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant leukemias; lymphocytic leukemias; plasmacytomas; leiomyosarcomas; and rhabdomyosarcoma.

In another embodiment of the invention, one or more of the Type II cells used in fusion can be tumor cells that are epithelial in origin (carcinomas) i.e. cancers of the following types: squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; transitional cell carcinomas; squamous cell carcinomas; choriocarcinomas; seminomas; embryonal carcinomas; malignant teratomas; and terato carcinomas.

In another embodiment of the invention, one or more of the Type II cells used in fusion can be leukemia cells e.g. cancers of the following types: acute lymphocytic leukemia and acute myelocytic leukemia (myeloblastic, promyeloblastic, myelomonocytic; monocytic, and erythroleukemia); chronic leukemia, e.g. cancers of the following types: e.g. (chronic myelocytic (granulocytic) leukemia and chronic lymphocytic leukemia); and polycythemia vera; lymphoma of types; (Hodgkin's disease and non-Hodgekin's disease), and multiple mycloma, Waldenström's macroglobulinemia, and heavy chain disease.

In another embodiment of the invention, one or more of the Type II cells used in fusion can be tumor cells, wherein the tumor cells are induced by chemical carcinogens or radiation. Chemical carcinogens comprise but are not limited to, carcinogens associated with cigarette smoking, such as hydrocarbons and carcinogenic air, food, cosmetics and other pollutants.

In another embodiment of the invention, one or more of Type II cells used in the fusion to generate heterohybrid cells are any cell containing and/or expressing misfolded proteins that causes a disease in the mammal. For example, but not limited to, disorders associated with protein aggregation and misfolded proteins, such as neurodegenerative diseases (e.g. Alzheimer's disease, Huntington's disease; Parkinson's disease; amyotrophic lateral sclerosis (ALS), polyglutamine diseases, CJD etc.), neoplastic disorders, cardiovascular disorders, skeletal muscle disorders, and other disorders associated with misfolded proteins well known to medical practitioners.

In another embodiment, the Type I or Type II cells used for the fusion can originate from eukaryotic or prokaryotic organisms. Cell fusion can comprise of any combination of prokaryotic cells, eukaryotic cells, eukaryotic cell lines and immortalized eukaryotic cell lines.

In another embodiment, one or more of the Type II cells used in fusion can be any infected cell, including whole tissues, isolated cells and immortalized cell lines infected or transformed with an intracellular pathogen and/or infectious agent. An intracellular pathogen is any viable organism, comprising but not limited to, viruses, bacteria, fungi, protozoa and intracellular parasites, capable of existing within a mammalian cell and causing a disease in the mammal. The cell can become infected occur prior to cell fusion, during cell fusion or of heterohybrid cells.

In another embodiment of the invention, one or more of the Type II cells can be infected with viruses comprising, but not limited to, hepatitis type A, hepatitis type B, hepatitis type C, influenzia, varicella, adenovirus, HSV-1, HSV-II, rinderpest rhinovirus, echovirus, retroviruses, rotavirus, respiratory syncytial virus, papilloma virus, papova virus, cytomegalovirus, echinovirus, abovirus, hantavirus, coxsackie virus, mumps virus, measles virus, rubella virus, polio virus, HIV-1, HIV-II, bird flu viruses and other viruses. In addition, cells can be transfected with a viral gene. Infection with the virus can occur prior to cell fusion, during cell fusion or of heterohybrid cells.

In another embodiment of the invention, one or more of the Type II cells used in the fusion can be bacteria-infected cells comprising of, but not limited to, bacteria causing tuberulosis, gonorrhea, typhoid, meningitis, osteomylitis, meningoccal, septicemia, endometritis, conjunctivitis, peritonitis, pyelonephritis, pharyngitis, septic arthritis, Celluloids, epiglottitis, salpingitis, otitis media, shigella dysentery, gastroenteritis, etc. Cells may also be infected with intracellular bacteria, comprising of, but not limited to, Mycobacteria, Rickettsia, Mycoplasma, Neisseria, and Legionella. Bacterial-infection can occur prior to cell fusion, during cell fusion or of heterohybrid cells.

In another embodiment of the invention, one or more of the Type II cells used in the fusion can be infected with intracellular protozoa comprising of, but not limited to, Leishmania, Kokzidioa, and Trypanosome. Furthermore, one or more of the cells used in the fusion can be infected with intracellular parasites comprising, but not limited to, Chlamydia and Rickettsia.

Method of Cell Fusion

One aspect of the present invention relates to the fusion of at least one Type I cell with at least one type II cell. Methods to fuse cells are commonly known by persons of ordinary skill in the art, and are disclosed in U.S. Patent Application 2006/0068495 which is incorporated in its entirety herein by reference.

In one embodiment, methods to generate a heterohybrid cell for use in the methods as disclosed herein include electrofusion. Briefly, the process of electrofusion comprises two steps: a creation of contact between the cells, and destabilization of cell membranes, which is called electropulsion (i.e. by applying an electric field). The contact step may take place before of after membrane destabilization due to the electric field, membrane destabilization necessitating control methods and flow of the field conditions.

The cells in contact in the time of the pulse can fuse during the process of spontaneous membrane repair or resealing. The efficiency of such fusion has been analyzed after low intensity alternating electronic field and have been applied for obtained hybridoma between B cells and myeloma cells.

Different protocols of electrofusion have been tested for the generation of hybrid cells, all characterized by an empirical manipulation (Scott-Taylor et al, 2000; Hayashi et al, 2002) which are useful in the methods of the present invention. In some embodiments, in order to establish a close cell-to-cell contact or alignment, a first dielecrrophorosis step can be performed. Dielectrophorosis leads to unspecific alignment of cells. In some embodiments, the formation of cellular heterohybrid is assisted by a bispecific ligand that attaches to each cell to be used in the generation of a heterohybrid cell, where non-covalent complexes formed between each cell to be fused (i.e. a non-covalent complex comprising a type I cell-bispecific ligand-type II cell complex), and then an electrical field is applied, which increases the probability of increasing the number of heterohybrid cells of interest rather than homohybrid cells. One can use such a method as described in U.S. Patent Application 2006/0068495 which is incorporated in its entirety herein by reference.

Among the numerous parameters for generation of heterohybrid cells of interest for the methods as disclosed herein, there is relative affinity of the bispecific ligand for each of the cells to be fused, the cellular concentration of each type of cell and the ligand concentration and the equilibrium constant, which refers to the concentration of the ligand needed to occupy half of the receptors present on the cells it forms non-covalent attachments with. In some embodiments, the ligand can be, for instance but not limited to, an antibody and the receptor can be, for example an antigen on the surface of the cell to be involved in fusion.

Other methods for cell fusion are known by persons of ordinary skill in the art, and can be used in the methods of the present invention, for example a method for non-linear amplitude dielectrophoresis waveform for cell fusion is disclosed in U.S. Pat. No. 6,916,656 which is incorporated herein by reference. There are a number of other different techniques (electrical, mechanical, chemical) that can be used to perform cell fusion, which are useful in the method of the present invention. Methods that involve electrical means can require a voltage waveform generator connected to an electrode device. Exemplary methods to perform electrical, mechanical, and chemical cell fusion are disclosed in the following U.S. patents and published PCT application, which are incorporated in their entirety herein by reference: U.S. Pat. No. 4,326,934; U.S. Pat. No. 4,441,972; U.S. Pat. No. 4,764,473; U.S. Pat. No. 4,784,954; U.S. Pat. No. 5,304,486; U.S. Pat. No. 6,010,613; WO 00/60065; WO03/102125; WO03/070933; WO03/035003; European Patent Applications 1734111 and 1509594 Further methods for cell fusion by non-linear dielectrophoresis waveform techniques for cell fusion are disclosed in the following U.S. Pat. Nos. 4,561,961; 5,001,056; 5,589,047; 5,650,305.

Resident time of the mixture inside the electrical field must be set in such amanner that whatever the form of the flow (continuous or sequential flow), it must last a time sufficient to allow accumulation of permeabilizing field conditions at surface of cells. The permeabilizing field is the field which induces a membrane potential difference locally greater than the value inducing a membrane destabilization. This parameter is a function of the size of the cell, of its shape, of its origin and of its physiological state. Permeabilizing field conditions result from the accumulation of positive and negative charges on opposite side of the surface of a cell due to the application of an electrical field.

If electrofusion of cells is used, the key parameters that must be determined to allow accumulation of permeabilizing field conditions at surface of cells are: i) intensity of electrical field, ii) number of pulses, iii) duration of each pulse, and. iv) speed of the flow when electropulsation (or electrofusion) is achieved on a continuous flow. Those parameters have to be ascertained for each type of cells to be fused, and a balance between parameters of each cell type is to be used to set the best conditions of electrofusion.

Setting the best value of the electrical field may be carried out by measuring percentage of permeabilized cells as a function of the electrical field value. The curves obtained allow one to determine electrical field value for which about 100% of cells are permeabilized (E100). Duration (t) and number (n) of pulses suitable for electrofusion are ascertained by measuring the percentage of permeabilized cells as a function of the duration of the pulses (nxt).

A means for measuring percentage of permeabilized cells as a function of any preceding parameters is to use propidium iodide (PI). PI is a fluorescent dye that enters only in dead cells and that binds on nucleic acid inside nucleus of cells. After addition of this fluorescent dye a short time (about 10 minutes) before eletropulsation, the number of permeabilized cells is evaluated by detecting presence of fluorescent cells. The detection may be carried out with a Fluorescence Activating Cell Sorter (FACS) for instance, and the level of fluorescence is measured in arbitrary unit. Low fluorescence (ranging from about 2 to about 4 U.A. for instance) is indicative of non-permeabilized cells, high fluorescence (ranging from about 600 to about 10000 U.A. for instance) is indicative of dead cells and intermediate fluorescence (ranging from about 70 to about 100 U.A. for instance) is indicative of permeabilized cells having loaded the fluorescent dye. Hence, a fluorescent level may be considered low when it is about 30 to about 50 times lower than the intermediate fluorescence, which is considered as intermediate when it is about 6 to about 10 times lower than the higher fluorescence. In case of sequential flow process, after having set the electric field intensity and the duration of pulses, cells are submitted to a given number of pulses of electric field, and the electrodes function best if orientated on parallel plates, parallel bars or parallel grids.

In case of continuous flow process, after having set the values of electrical field intensity, the pulse duration, the frequency of delivery is determined in order for the cells to be submitted to a given number of pulses at the chosen speed of the flow. All these parameters and definitions are well known from the man skilled in the art. The speed of the flow for electrofusion may be for example of about 10 ml/min, and may be adapted according to the number of cells to be treated or to other necessities.

The flow rate is governed by the speed of the flow and the cross section of the pulsing chamber and may be adapted according to the number of cells to be treated or to other necessities (such as pulse frequency). It may be ranging from about 2 to about 75 ml/min in one pulsing chamber and is more preferably of about 10 ml/min. This allows the treatment of a larger volume of mixture than when applying the electrical field to a batch of medium containing cells. The speed of the flow and the resulting volume of cells exposed to electropulsation determine the number of fused cells. This is in contrast with the state of the art where the size of the electrofusion chamber in practice is inferior to 10 ml and thus severely limits the amount of produced heterohybrids.

In the state of the art, when HSPs and HSP complexes are obtained from the heterohybrids as disclosed herein for use to administer to a subject in order to elicit an immune response, the HSPs can be obtained from one or several millions of heterohybrids as disclosed herein. The preparation of many doses HSPs and HSP complexes of efficient quantity obtained from any specific heterohybrid.

In some embodiments, the cells for use in the generation of the heterohybrid cells for used in the methods as disclosed herein can be have their proliferation blocked or be killed before fusion with another type of cell. Treatment to kill or block proliferation can be performed on the cells before formation of heterohybrid cells, or after formation of the heterohybrid cells. One can use a treatment using any protocol known by persons of ordinary skill in the art and, such as, for example, gamma-irradiation, at doses preferably comprised between about 150 and 200 Gray (Habal et al., 2001, Kikuchi et al., 2001). Cells may also be rendered non-tumorigenic by the appliance of an electrical field in which the duration, the frequency and the intensity of the electrical impulsion are designed to kill cells.

In a particular embodiment of the invention, the intensity of the electrical field applied to the cells is comprised from about 100 to about 40.00 V/cm, and preferably from about 800 to about 2000 V/cm, and preferably from about 1200 to about 1800 V/cm and, more preferably from about 1400 to about 1600 V/cm. These values correspond to electrical field designed for the destabilization of the cell membrane; a higher intensity is required for killing cells. In an other embodiment of the invention, the duration of the electrical impulse is comprised from about 1 microsecond to about 100 millisecond, and preferably from about 20 to about 1000 microseconds, and preferably from about 50 to about 250 microseconds, and more preferably about 200. In another embodiment of the invention, the number of electrical impulses applied to a cell is comprised from about 1 to 100, and preferably from about 4 to 20, and more preferably about 5.

There is no need for the addition of any component, such as sucrose or other, that would be required to maintain cell integrity. This limits any potential external contamination and limits the number of components to be controlled and analyzed by regulatory authorities, and simplifies the process by using a fusion medium identical to the culture medium used for the preceding steps. As an example, AIM V medium (Gibco-Life Technologies) may be used. Therefore, it has a positive effect on fusion yield while preserving cell viability.

Methods for electrofusion can use electrical impulses that are unipolar or bipolar. In a more particular embodiment of the invention, the shape of the electrical impulse usable for the method is a square wave, a sinusoid, a triangle, or present exponential decline.

After being submitted to the process of electropulsation, cells present at their surface structural alterations resulting from destabilization, which spontaneously reseal. When two cells are brought close one to another, the step of resealing the structural alterations leads to the fusion of the two cells one with another. Hence, after going through the electropulsing, the sample in which the cells are in is left for a sufficient period of time at rest in order to allow the heterofusion to be achieved with high yield.

Means for measuring required time for resealing is to use propidium iodide (PI). This fluorescent dye is added at increasing time (ranging from 0 to 20 minutes for instance) after the last pulse, and the decreasing number of fluorescent cells in course of time is an index of the time required for resealing. The time needed for resealing all permeabilized cells may vary from one cell type to another cell type and is depending of the temperature at which the experiments are conducted (the more temperature decreases, the more time of resealing increases). The electrical treatment brings a local membrane fusion (membrane coalescence). The mixture must then be incubated at about 30 to about 37° C. during about 30 minutes to about 4 hours and more preferably about 1 hours to obtain a cellular fusion, i.e. hybrid cell formation.

### Characterization of Heterohybrid Fused Cells

Heterohybrids may be isolated from their other components, and particularly non-fused cells, and recovered from successive stems selecting on the presence of a marker specific for each type of cell, the first type of cell and the second type of cell. This can be done by any method known by the person skilled in the art, such as FACS sorting, differential centrifugation or magnetic bead selection.

In some embodiments, the cell population is characterized in that it contains from about 60 to about 100% of living cells. Cell viability may be characterized by cells permeability to a component such as Trypan blue or propidium iodide, or by assessing the functionality of cellular esterases. The cell preparation may be used as such. Another possibility is to freeze the cell preparation in order to store it before use. All methods and device to freeze, store and thaw cells are known to persons of ordinary skill in the art.

In some embodiments, the percentage of heterohybrids is comprised between about 15 and about 80% of the recovered cells, and preferably from about 25 to about 50% of the recovered cells. Heterohybrids can be observed and quantified by any method known to persons of ordinary skill in the art, such as incubation with fluorescent antibodies specific for each of the two initial types of cells and microscopic observation, or by FACS sorting. If a bispecific ligand was used in the process of cell fusion, some cells in the cell population can also contain cellular conjugates in which cells are still linked by the bispecific ligand.

The calculation of the percentage of heterohybrids is based on the number of heterohybrids in the cell population after fusion related to the number of cells initially present before fusion. In particular, under the assumption that only bikaryons are formed, a usable mathematical formula can be: (2N2)/(N1+2N2)*100, with N1=number of cells with one nuclei, N2: number of cells with 2 nuclei.

To characterize a heterohybrid cell to determine if it comprise fusion of the cells of interest (e.g. the heterohybrid cell is a DC-tumor heterohybrid cell) and therefore is useful for extracting HSPs and HSP complexes for use in the methods as disclosed herein, one can determine the expression of surface markers on the surface on the outside of the heterohybrid cell. In some instances the surface markers is an antigen on the cell surface. In particular, if a cell co-expresses at least one surface markers expressed specifically on type I cells (for example DC cells) that is not expressed on Type II cells, and at least one surface marker expressed specifically on Type II cells (i.e. tumor cells), that is not expressed on the Type I cells, then the cell is identified as a heterohybrid cell from the fusion of a Type I cell with a Type II cell.

Accordingly, if a heterohybrid cell comprises an antigen specific to one of the types of cells used in the fusion (i.e. an antigen specific to a type I cell, such as a DC) and also comprises an antigen specific to the other type of cell used in the fusion (i.e. an antigen specific to a type II cell, such as a tumor cell), then the heterohybrid is useful for extracting HSPs and HSP complexes for use in the methods as disclosed herein.

In some embodiments, if one of the types of the cell (e.g. a Type I cell) used in the generation of the heterohybrid cell is an antigen presenting cells, such markers or antigens that are specific to antigen presenting cells are, for example, but not limited to, IgG Fc receptors such as FcγRI (CD64), FcγRI (CD32), FcγRIII (CD16). In some embodiments, cell surface markers for DC are, for example but not limited to CCR1, CCR2, CCR5, CCR6, CXCR1, CCR7, TRANCE R/RANK, OX40L, LFA-1, ICAM-1, LFA-3, CD44, ICAM-3, DC-SIGN, CXCR4, CCR7, MDC/CCL22, TARC/CCL17, PARC/CCL18, CD91, DC-LAMP, B7-1/CD80, B7-2/CD86, CD47, LR3, DORA, ILT-3; FcγR, FcγRI, DEC-205, DCIR, dectin-2, CLEC-1, MR and can be used to identify heterohybrid cells as a result of the fusion of a DC cell with another cell. In some embodiments, due to its higher affinity for Fc, FcγRI (CD64) is a useful cell surface marker for heterohybrids that have been generated from the fusion of an antigen presenting cells with another cell. FcαR (CD89) surface marker of APCs also represents a possible surface marker for identifying heterohybrids that have been generated from the fusion of an antigen presenting cells with another cell. In some embodiments, a DC-sign receptor and the mannose receptors are also useful surface markers for identifying heterohybrids as a result of the fusion of a DC with another cell. Other possible surface markers for antigen presenting cells are CD83, CD80, or CD86 molecules, as well as adherence molecules such as ICAM-1 (CD54) or LFA 3 (CD-58), which can be used to identify heterohybrids that have been generated from the fusion of an antigen presenting cells with another cell. In some embodiments, the antigen presenting cell can be identified using antibodies to the markers, such as for example but not limited to, anti-MHC class II (M5/114), CD86 (GL1), B7-DC (TY25), CD40 (3/23) or ICAM (3E2) antibodies, as disclosed in the Examples.

In some embodiments, where the type of the cell (e.g. a Type II cell) used in the generation of the heterohybrid cell is a tumor cell, such cell-surface markers useful in identifying a heterohybrid cell as a result of the fusion of a tumor cell with another cell can be antigen-associated markers or antigens specific to tumor cells, for example, but not limited to, prostate PSA or PSMA, Her-2/neu, HSP-70, HER2/Her-2, BRAC1, BRAC2, Rb, p53, EGF-R, MUC-1, Melan-A, MART1, p53, CEA, NYESO, TRP-1, TRP-2, MAGEs, BAGEs, TAG72, GP-100; sialyl-Lewis oligosaccharides, selectine ligands, such as O-glycanes or gangliosides, which are glycanic antigens associated to cancer, in particular LH or MUC-1 antigens, and are useful to identify heterohybrid cells that have been generated from the fusion of an tumor cell with another cell. In other embodiments, viral antigens expressed by tumor cells, such as HBs, HCV, papillomavirus, HPV E6, E7 can also be used to identify heterohybrid cells that have been generated from the fusion of a tumor cell with another cell.

In alternative embodiments, one can perform morphological analysis to characterize a cell to determine if it is heterohybrid cell as a result of the fusion of at least two cells of interest (e.g. the cell is a DC-tumor heterohybrid cell) and therefore is useful for extracting HSPs and HSP complexes for use in the methods as disclosed herein. Such morphological analysis can be performed by any method commonly known by persons of ordinary skill in the art, for example but not limited to electron microscopy. If on morphological analysis, a cell comprises the genetic material of more than one cell, for instance the cell has two nuclei or more, the cell is a result of a fusion of two cells. Alternatively, if a cell comprises one nuclei but the nuclei is larger than in normal cells at the same type of cell and in the same phase of cell cycle, it is likely that the large nuclei is a result of the fusion of two nuclei to form one large nuclei, and such a cell comprising a fused nucleic is commonly referred to in the art as a tetraploid cell and is a result of the fusion of at least one cell with another cell. Methods for performing electron microscopy are commonly known by persons of ordinary skill in the art and are encompassed for use in the methods as disclosed herein.

Method of Recovering HSPs and HSPs from Heterohybrid Cells

Another aspect of the present invention relates to recovery of HSPs and HSP complexes, and aggregates thereof from heterohybrid cells, such as DC-tumor heterohybrid cells as disclosed herein.

Heat shock proteins (HSPs) are cellular chaperone proteins. The term “chaperone protein” as used herein refers to, in general, any protein that facilitates intracellular protein folding and/or facilitates the processing and functioning of other cellular proteins. Such processing comprises of, but is not limited to, activities such as folding, unfolding, refolding, stabilizing, oligomerizing, salvaging, scavenging and discarding cellular proteins during normal intracellular activities. Chaperones are generally capable of associating with other molecules, such as other chaperones and/or other proteins and/or peptides to form chaperone protein-protein complexes.

In some embodiments, the HSPs and HSP complexes recovered from heterohybrid cells such as DC-tumor heterohybrid cells can be chaperone and chaperone protein complexes, for example HSP and HSP complexes for example, but not limited to, gp96, heat shock proteins such as HSP110 and HSP110 isoforms, HSP90 and HSP90 isoforms HSP86 and HSP84, HSP70 and HSP70 isoforms HSP70, HSP78, HSP75, and HSC70, HSP60, HSP40, the small heat shock protein family (sHsp); HSP27 and HSP20 and other sHSPs, and calreticulin (CRT) and complexes thereof. In some embodiments, HSP complexes useful in the methods here can be present in aggregates or in multimeric forms.

In some embodiments, the HSPs and HSP complexes recovered from heterohybrid cells, such as for example DC-tumor heterohybrid cells can be used to prepare a pharmaceutical composition, for example a vaccine composition.

In specific embodiments, the HSPs and HSP complexes recovered from heterohybrid cells, such as for example DC-tumor heterohybrid cells comprise members of the HSP70 superfamily, for example, but not limited to, HSP70-1a, HSP701b, HSP70-HOM, HSP70-4, HSP70-5, HSP70-9b, HSP75 and HSC70 and homologues, isoforms and derivatives thereof.

One aspect of the present invention relates to HSPs and HSP complexes recovered from heterohybrid cells, such as for example DC-tumor heterohybrid cells that comprise an antigen. In some embodiments, the antigen is a cellular protein or fragment thereof from the heterohybrid cell, for example a tumor antigen or tumor-associated antigen from a heterohybrid cell such as a DC-tumor heterohybrid cell. Tumor antigens are commonly known by persons of ordinary skill in the art and any tumor antigen known is useful in the methods as disclosed herein.

In some embodiments, the HSPs and HSP complexes useful in the methods as disclosed herein comprise co-factors and accessory proteins. Co-factors and accessory proteins to HSPs are among the most highly conserved proteins in existence. For example, DnaK, the HSP70 from E. Coli has about 50% amino acid sequence identity with HSP70 proteins from eukaryotes. The HSP60 and HSP90 families also show similarly high levels of intrafamilies conservation (Hickey, et al, 1989, Mol Cell Biol. 9:2615; Jindal (1989) Mol Cell Biol, 9:2279). Accordingly, as HSPs are so highly conserved, the methods as disclosed herein are also applicable to homologous HSPs and HSP co-factors and accessory proteins in any species of organisms including bacteria, fungi, plants and animals. In one embodiment, the HSPs and HSP complexes useful in the methods as disclosed herein are mammalian HSPs and HSP complexes, and in particular embodiments, the HSPs and HSP complexes are human HSPs and HSP complexes. In some embodiments, the HSP complexes as disclosed in the methods herein comprise antigens and HSP co-chaperones, for example but not limited to Hip, Hop, Hup, Hap and CHIP co-chaperones.

Without being bound by theory, many heat shock proteins function together in co-chaperone complexes, such as Hsp70/Hsp40 that along with GrpE, acts as an ATP-regulated shuttle complex for newly synthesized proteins. Many of these nascent peptides are delivered to Hsp90-containing complexes, which play a critical role in the stabilization and activation of key signaling kinases and hormone receptors. The Hsp60/Hsp10 complex forms an alternative protein folding mechanism in the mitochondria. Small heat shock proteins including Hsp27 and the crystallins (α- and β-crystallin) form large oligomeric complexes that function to prevent protein aggregation.

The Hsp70 family of heat shock proteins contains multiple homologues ranging in size from 66 kDa to 78 kDa. The most studied include the cytosolic stress-induced Hsp70 (Hsp72), the constitutive cytosolic Hsc70 (Hsp73), and the ER-localized BiP (Grp78). Hsp70 family members contain a highly conserved N-terminal ATPase domain, as well as a conserved C-terminal hydrophobic peptide binding domain (PBD) and more variable alpha-helical “lid” domain. ATP-bound Hsp70 freely associates with nascent or misfolded peptides (open lid), causing a conformational change that activates inherent Hsp70 ATPase activity and enhances the association with Hsp40 to further accelerate conversion to an ADP-bound (closed lid) form. Co-chaperones such as Hip, Hop, Hup, Hap and CHIP, modulate Hsp70 nucleotide exchange and substrate binding to coordinate the folding of newly synthesized proteins, re-fold misfolded or denatured proteins, coordinate trafficking of proteins across cellular membranes, disassemble clathrin-coated vesicles, inhibit protein aggregation, and target the degradation of proteins via the proteasomal pathway

In some embodiment, one or more HSP and/or HSP complex is recovered from a heterohybrid cell, such as for example DC-tumor heterohybrid cell. Methods to recover HSPs and/or HSP complexes from a biological sample comprising heterohybrid cells are commonly known by persons of ordinary skill in the art, and include but are not limited to methods such as immunoprecipitation and/or ADP-affinity chromatography, GST-fusion pull-down, sequential ATP agarose affinity chromatography, DEAE cellulose and Sephadex G25 chromatography.

In one embodiment, HSPs and/or HSP complexes are recovered from a heterohybrid cell, such as for example DC-tumor heterohybrid cell by immunoprecipitation. Methods for immunoprecipitation of HSP and HSP complexes are disclosed in the Examples and are commonly known by persons of ordinary skill in the art. In some embodiments, immunoprecipitation can be performed using commercially available kits, or can be performed by those of skill in the art as disclosed in the Examples. Commercial antibodies that can be used in the method as disclosed herein are available from companies such as Stressgen, Sigma, abchem, Pharmigen, chemicon, calbiochem etc. As an illustrative example but not by way of limitation, antibodies from Stressgen that are useful in the methods as disclosed herein include, for example, anti-HSP25 (SPA-801) anti-HSP40 (SPA-450), anti-HSP90 (SPA-830) or anti-HSP110 (SPA-1103). In one embodiment, an antibody useful to immunoprecpitiate a HSP protein or HSP complex from a heterohybrid cell the Ab46 antibody against HSP70 as disclosed in the Examples.

In one embodiment, a biological sample comprising heterohybrid cells such as DC-tumor heterohybrid cells is a cell lysate. Such a lysate can be prepared by mechanical means, or by contacting the cells with hypertonic buffer, a hypotonic buffer or detergent. In some embodiments, the biological sample can also comprised of a solution containing a purity of proteins, including but not limited to HSP and/or HSP complex.

In another embodiment, the HSPs and HSP complexes recovered from heterohybrid cells, such as for example DC-tumor heterohybrid cells can be modified by other processes known to persons of ordinary skill in the art to further amplify the immunogenicity of the HSPs and HSP complexes.

In another embodiment, HSPs and HSP complexes recovered from heterohybrid cells, such as for example DC-tumor heterohybrid cells and can be enriched, but not purified to homogenicity.

In some embodiments, the HSPs and HSP complexes recovered from heterohybrid cells, such as for example DC-tumor heterohybrid cells are purified, but not to the extent that the extracted complex excludes the desired antigenic determinants to be used to elicit an immune response. Such a desired antigenic determinant, is for example, is an immune response to a cancer cell one wishes to reverse immune tolerance to, or an antigen one wishes to stimulate activation of antigen specific CD4⁺ and CD8⁺ cells.

In another embodiment, the HSPs and HSP complexes recovered from heterohybrid cells, such as for example DC-tumor heterohybrid cell can be recovered after they are released from the heterohybrid cells into the cells' surroundings. Accordingly, HSPs and HSP complexes can be recovered from biological samples, such as for example, but not limited to, bodily fluids, secretions, culture supernatants, fermentation broth and other sources known to persons of ordinary skill in the art.

Uses of HSPs and HSP Complexes from Heterohybrid Cells

In one embodiment, the HSPs and HSP complexes recovered from heterohybrid cells, such as for example DC-tumor heterohybrid cells can be used to prepare a pharmaceutical composition, such as a vaccine, which can be administered to a subject to elicit an immune response. In some embodiments, the pharmaceutical composition comprises HSPs and HSP complexes recovered from heterohybrid cells, such as for example DC-tumor heterohybrid cells alone or in combination with other proteins. In some embodiments, the HSPs and HSP complexes recovered from heterohybrid cells, such as for example DC-tumor heterohybrid cells can be used to make such compositions for administration to a subject in need of treatment or prevention of a disease.

Depending on the disease, the HSPs and HSP complexes recovered from heterohybrid cells can be recovered from a lysate of heterohybrid cells that are generated from the fusion of a cancer tissues, tumor cells, infected cells from biopsies, or a transformed or transfected cells from cell cultures with another cells, such as a antigen presenting cell or a DC.

In some embodiments, the HSPs and HSP complexes recovered from heterohybrid cells, such as for example DC-tumor heterohybrid cells can be used in compositions can be used to treat or prevent cancer, or any other malady or infectious disease.

As described herein, the HSPs and HSP complexes recovered from heterohybrid cells can stimulate CD4⁺ and CD8⁺ T cells, antigen specific CD4⁺ and CD8⁺ T cells, reverse immune tolerance etc. In one embodiment, the HSPs and HSP complexes recovered from heterohybrid cells comprise an antigen which triggers an immune response. In some embodiments, the heterohybrid cell useful in the methods as disclosed herein is generated from the fusion of a Type I with a Type II cell, where either the Type I and/or Type II cell has had an antigen introduced. Introduction of an antigen can occur to a Type I and/or Type II cell before fusion or a fused heterohybrid cell such as a DC-tumor heterohybrid cell to stimulate any of the following; CD4⁺ T cells, CD8⁺ T cells, specific antigen CD4⁺ T cells, specific antigen CD8⁺ T cells and any other cells that stimulate a CTL response.

In further embodiments, the HSPs and HSP complexes recovered from heterohybrid cells used to stimulate and immune response, such as stimulation of CD4⁺ and CD8⁺ T cells can be used in a pharmaceutical composition. In some embodiments, such a pharmaceutical composition comprises HSPs and/or HSP complexes and an antigen, where the antigen triggers an immune response. In some embodiments, the HSP and/or HSP complex is a HSP70 or HSP70 complex, or aggregates thereof.

In another embodiment a pharmaceutical composition useful in the methods as disclosed herein is prepared by combining HSPs and/or HSP complexes recovered from one population of heterohybrid cells (such as DC-tumor cells) with one or more HSPs and/or HSP complexes recovered from a different population of heterohybrid cells. By way of a non-limiting example, HSPs and/or HSP complexes recovered from a DC-breast tumor heterohybrid cell can be combined with HSPs and/or HSP complexes recovered from a DC-colon tumor heterohybrid cell, or DC-melanoma heterohybrid cell. Any number of combinations of HSPs and/or HSP complexes recovered from different heterohybrid cell populations can be used, where the heterohybrid populations have at least one different Type I cell and/or Type II cell.

In some embodiments, the pharmaceutical compositions comprising HSPs and HSP complexes recovered from heterohybrid cells can comprise HSPs and HSP complexes or aggregrate thereof, of any of the following HSPs for example; calreticulin (CRT), GRP94/gp96, gp96, grp75/mt, BiP/grp 78, HSP90, HSP 72, HSP 60, HSP 70, HSC70, HSP40, HSP 27 or homologues or fragments or variants thereof.

In one embodiment, the HSPs and/or HSP complexes recovered from heterohybrid cells, such as DC-tumor cells, are useful for treating and/or preventing cancer in a subject, where the method comprises administering to a subject a therapeutically or prophylatically effective amount of a pharmaceutical composition as disclosed herein, where the pharmaceutical composition comprises HSPs and/or HSP complexes recovered heterohybrid cells, such as DC-tumor cells. The HSPs and/or HSP complexes can be recovered from heterohybrid cells that have been generated from the fusion of Type II cell which is a cancer cell of the type of cancer in the subject, or metastasis thereof, with a Type I cell.

In another embodiment, the pharmaceutical composition as disclosed herein is useful in methods to treat and/or prevent a disease caused by an infectious agent, e.g. a virus, a bacterium, or a parasite, in a subject, wherein the pharmaceutical composition comprises HSPs and/or HSP complexes recovered from heterohybrid cells that have been generated from the fusion of Type II cell which is a cell that expresses infectious agent, e.g. the antigenic molecule displaying antigenecity of an antigen of an infectious agent, with a Type I cell.

In one embodiment, the pharmaceutical composition as disclosed herein is useful in methods to stimulate CD4⁺ and/or CD8⁺ T cells and/or treating and/or preventing an infectious disease in a subject by administering to said subject a therapeutically or prophylatically effective amount of pharmaceutical composition as disclosed herein. In some embodiments, the pharmaceutical composition comprises HSPs and/or HSP complexes recovered from heterohybrid cells that have been generated from the fusion of Type II cell which is a cell that expresses infectious agent, e.g. the antigenic molecule displaying antigenecity of an antigen of an infectious agent, with a Type I cell.

In another embodiment, the pharmaceutical composition as disclosed herein is useful in methods to elicit an immune response against an antigen in a subject, the method comprising administering to a subject a therapeutically or prophylatically effective amount of pharmaceutical composition comprising of HSPs and/or HSP complexes recovered from heterohybrid cells.

In some embodiments, the pharmaceutical compositions or vaccine as disclosed herein can be administered to a subject by any pharmaceutically acceptable route and in any pharmaceutically acceptable form, for example but not limited to various galenic forms such as intrademal, subcutaneous, intravenous, intralymphatic, intranodal, intramucosal or intramuscular administration.

In some embodiments, the pharmaceutical composition as disclosed herein can be prepared as a vaccine. In some embodiments, such a vaccine is useful in the treatment and/or prevention of a variety of diseases, for example but not limited to, cancer, viral diseases, infections or any other disease identified by persons or ordinary skill in the art whereby the pharmaceutical composition as disclosed herein could be used.

In some embodiments, the pharmaceutical composition as disclosed herein can be used in conjunction with other treatments, for example other anti-cancer therapies such as, for example, chemotherapy (for example treatment with cisplatin or 5 FluoroUracil), radiotherapy, hormones therapy, and treatment with anti-angiogenic agents and drugs.

In some embodiments, the pharmaceutical composition as disclosed herein can comprise about 30%, 20%, 10% or 5% (by dry weight) heterologous protein (also referred to herein as “contaminating protein”) other than the HSPs and/or HSP complexes recovered from the heterohybrid cells.

In further embodiments, the HSPs and/or HSP complexes recovered from the heterohybrid cells such as DC-tumor heterohybrid cells can be linked to a carrier molecule, such as, for example but not limited to a carrier molecule which increases the immunogenic property of the HSP and/or HSP complex, or a carrier molecule facilitates the uptake of HSPs and/or HSP complexes by a phagocytotic cell. As a non-limiting example, carrier molecules can be natural or synthetic biodegradable microparticles. In some embodiments, HSPs and/or HSP complexes can be associated, such as non-covalent conjugation or covalent conjugation to the carrier molecule, such as a surface of a microparticle.

In some embodiments, the pharmaceutical composition as disclosed herein is useful in methods to stimulate CD4⁺ and CD8⁺ T cells, stimulate antigen specific CD4⁺ and CD8⁺ T cells, reverse the immune tolerance of cells such as cancer cells, and for administration to a subject for the treatment and/or prevention of diseases, such as cancer, infectious diseases, and autoimmune diseases. In some embodiments, the pharmaceutical composition as disclosed herein comprises HSPs and/or HSP complexes recovered heterohybrid cells, where the heterohybrid cells can be generated from the fusion of at least two cells where the cells can be any combination of autologous or allogenic to the subject being administered the pharmaceutical composition.

In some embodiments, the pharmaceutical composition as disclosed herein can be used as vaccines, for example vaccine that are suitable for administration to humans, as well as veterinary uses.

In further embodiments, the pharmaceutical composition as disclosed herein can be administered to any mammal including, but not limited to, domestic animals, such as cats and dogs, wild animals including foxes and raccoons; livestock and fowl, including horses, cattle, sheep, turkey and chickens. In further embodiments, the pharmaceutical composition as disclosed herein can be administered to rodents.

### EXAMPLES

The examples presented herein relate to the methods and compositions of heat shock proteins and heat shock proteins recovered from a heterohybrid cell, wherein the heterohybrid cell is a fusion of at least one antigen presenting cell and at least one tumor cell to induce a CD4⁺ or CD8⁺ immune reaction. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods

Mice and cells C57BL/6 wild-type (WT) mice were obtained from The Jackson Laboratory (Bar Harbor, Me.). MUC1 transgenic mice on C57BL/6 background were obtained from Dr. S. J. Gendler (Mayo Clinic, Scottsdale, Ariz.) (25). Polymerase chain reaction (PCR) was performed routinely to identify MUC1 Tg-positive mice in the colony. MyD88 knockout (MyD88 KO) mice were obtained from Dr. Stuart Levitz (Boston Medical School, Boston, Mass.). The mice were maintained in microisolator cages under specific pathogen-free conditions.

Murine MC38 colon adenocarcinoma cells were stably transfected with a MUC1 cDNA, resulting in MC38/MUC1 (26, 27). DC were generated from bone marrow cultures of C57BL/6 WT mice using the method previously described (28). DC were fused to MC38/MUC1 cells using previously described method (29, 30). The fusion efficiency of DC-tumor fusion cells (FC/MUC1) was around 20-30%.

Immunoprecipitation of HSP70-associated complexes from DC-tumor fusion cells. MC38/MUC1 tumor cells and FC/MUC1 (30, 31) were incubated in lysis buffer (PBS, 1% Triton X-100, 1 mg/ml BSA, 0.2 U/ml Aprotinin. 1 mM PMSF) with 20 U/ml Apyrase (Sigma) for 1 h on ice (Apyrase is used to deplete ATP in the extracts and stabilize HSP70 associations). The lysates were clarified by centrifugation, and the aqueous phase were collected. The Ab46 antibody against HSP70 (32) at concentration of 1:100 were added and incubated overnight at 4° C. Then, 50 μl of protein A/G (1:1) agarose were added and incubated at 4° C. for additional 1 hour. After extensive wash with lysis buffer, the immunoprecipitates were eluted with PBS with high salt (250 mM NaCl). The elute of HSP70.PC-F (derived from DC-tumor cells) or HSP70.PC-Tu (derived from tumor cells) were diluted to bring the salt concentration to 150 mM, quantified and aliquoted at 100 μg/ml for in vivo immunization. For protein analysis, the immunoprecipitates were dissolved in SDS sample buffer (0.1 Tris-Cl, 4% SDS, 20% glycerol, 0.05% bromphenol blue, 5% 2-ME), and analyzed by immunoblotting. For in vivo and in vitro experiments, the preparations were routinely checked by limulus amebocyte lysate (LAL kit, Cambrex Bio Science Inc., Walkersville, Md.) assay to ensure no contamination of endotoxin.

Immunoblotting The proteins were subjected to SDS-PAGE and transferred on nitrocellulose membrane. The membranes were incubated with anti-HSP25 (SPA-801) anti-HSP40 (SPA-450), anti-HSP90 (SPA-830) or anti-HSP110 (SPA-1103) antibodies (Stressgen, Victoria, BC, Canada) or anti-MUC1 antibody (HMPV, BD Pharmingen, San Diego, Calif.) and antigen/antibody complexes were visualized by enhanced chemiluminescence (ECL detection system, GE).

In vivo tumor rejection Seven-week-old WT mice were immunized subcutaneously (sc) with 1.5 or 3 μg of HSP70.PC-F or HSP70.PC-Tu on Day 0 and Day 7. A group of mice immunized with 2×10⁵ FC/MUC1 or injected with PBS were used as controls. On Day 14, the mice were challenged by subcutaneous injection in the flank with 2×10⁵ syngeneic MC38/MUC1 tumor cells. In the separate experiment, MUC1.Tg mice (6/group) were immunized twice with 1.5 μg HSP70.PC-F or HSP70.PC-Tu and then challenged with 2×10⁵ tumor cells in both sides of the flank of the mice one week after second immunization. All the mice were followed for 30 days to determine the tumor incidence. Tumor volume was measured by caliper for twice a week, and tumors with a diameter of >2 mm were designated as positive.

CTL assay CTL assay was performed as described previously (33, 34). Briefly, splenocytes were isolated from vaccinated or control mice. MC38/MUC1 tumor cells (1−2×10⁶ cells) were labeled with 100-200 μCi Na₂⁵¹CrO₄ for 60 min at 37° C., and then washed to remove unincorporated isotope. Splenocytes or tumor targets were resuspended in culture medium (RPMI-1640 medium supplemented with 15 mM HEPES (pH 7.4), 5% heat-inactivated-FCS, 2 mM L-glutamine, 0.1 mM nonessential amino acids, 1 mM sodium pyruvate, 100 U/ml penicillin, 100 μg/ml streptomycin, and 5×10⁻⁵ M β-mercaptoethanol), and then combined at various effector-to-target ratios (E:T) in 96-well V-bottom plates. In the antibody-blocking assay, the target cells were incubated with anti-MHC class I mAb (M1/42.3.9.8) before addition of the effector cells. The plates were centrifuged at 100×g for 5 min to initiate cell contact and incubated for 5 h at 37° C. with 5% CO₂. After incubation, supernatants were collected and radioactivity was quantitated in a gamma counter. Spontaneous release of ⁵¹Cr were determined by incubation of targets in the absence of effectors, and maximum or total release of ⁵¹Cr by incubation of targets in 0.1% Triton X-100. Percentage of specific release of ⁵¹Cr is calculated by the following equation: percentage specific release={(experimental−spontaneous)/(maximum−spontaneous)}×100.

T cell proliferation Splenocytes and/or lymph node cells (LNC) were isolated from naïve mice or mice immunized with HSP70.PC-F or HSP70.PC-Tu. Erythrocytes and dead cells were removed by centrifugation over a Ficoll-Hypaque gradient. The cells were washed and resuspended at the appropriate concentration in RPMI-1640 medium supplemented with 15 mM HEPES (pH 7.4), 5% heat-inactivated-FCS, 2 mM L-glutamine, 100 U/ml penicillin, 100 μg/ml streptomycin, and 5×10⁻⁵ M β-mercaptoethanol. Cells were incubated in the presence or absence of HSP70.PC-F or HSP70.PC-Tu as indicated concentration. Incubations were performed in 96-well, U-bottom plates for 5 days. T-cell proliferation were assessed by [³H] thymidine incorporation after an additional 12-h incubation with 1 μCi/well of [³H] thymidine.

Phenotype of T cells MUC1.Tg mice were immunized subcutaneously (sc) on Day 0 and Day 7 with 1.5 μg of HSP70.PC-F or HSP70.PC-Tu in posterior flank near the base of tail. One week after second immunization, mice were sacrificed and inguinal LN collected and stained with anti-CD4 (L3T4), CD8 (Ly-2), IFN-γ (XMG1.2), CD69 (H1.2F3), CD44 (IM7) and/or IL-15R (TM-β₁) antibodies (BD Pharmingen) and analyzed by flow cytometry using CellQuest software (BD Biosciences).

DC generation and maturation DC were generated using the method described previously (28) with minor modification. Briefly, bone marrow cells were selected by lysis of red cells and depletion lymphocytes and Ia+ cells by series of treatments with panels of mAbs followed by rabbit complement, and then cultured in the presence of GM-CSF (20 ng/ml; Sigma). On third day of culture, the non-adherent cells were collected and cultured in medium containing GM-CSF for O.N. Then the loosely adherent DC were collected and cultured in the presence or absence of HSP70.PC-F or HSP70.PC-Tu (10 μg/ml) for 6 or 24 hours in 24 well plate. The DC were collected, stained with anti-MHC class II (M5/114), CD86 (GL1), B7-DC (TY25), CD40 (3/23) or ICAM (3E2) antibodies (BD Pharmingen) and analyzed by FACS. In a separate experiment, immature DC were co-cultured with 10 μg/ml LPS (Sigma, St. Louis, Mo.), HSP70.PC-F, boiled HSP70.PC-F, HSP70.PC-Tu, HSP70-PC derived from fusions of MC38/MUC1 tumor cells in the presence of Polyethylene glycol (PEG, Sigma) or the mixture of DC and MC38/MUC1 tumor cells, and then analyzed for the expression of indicated molecules.

To determine the DC maturation in vivo, inguinal LN were obtained from mice immunized twice with 1.5 μg of HSP70.PC-F, HSP70.PC-Tu or injected with PBS and snap frozen. Cryosections were obtained, stained with anti-CD86 mAb and then examined under microscopy:

HSP70.PC pull down from fusion cells by GST-HSP70 fusion proteins MC38/MUC1 carcinoma cells were transfected with a total of 7.5 μg of pCMG plasmid vector encoding GST-HSP70 fusion proteins per 100 mm diameter dish using Lipofectamine (Invitrogen, Carlsbad, Calif.). The transfected MC38/MUC1 were fused with DC. The cells, 2×10⁷ each, were lysed in lysis buffer (50 mM Na₂HPO₄, 1 mM sodium pyrophosphate, 20 mM NaF, 2 mM EDTA, 2 mM EGTA, 1% Triton X-100, 1 mM dithiothreitol, 200 μM benxamidine, 40 μg/ml leupeptin, 300 μM phenylmethylsulfonyl fluoride). The detergent-solublized cell lysates were incubated with glutathione sepharose beads O.N. at 4° C. Beads were washed twice in lysis buffer and twice in lysis buffer with 150 mM NaCl. Proteins bound to the glutathione sepharose beads were eluted with glutathione elution buffer (50 mM tris pH 8.5 and 20 mM reduced glutathione).

Detection of MUC1 peptide from GST-pull down HSP70.PC The GST-pull down of HSP70.PC-F or HSP70.PC-Tu or purified HSP70.PC-F or HSP70.PC-Tu were pre-coated the ELISA plates (Nunc, Naperville, Ill.) ON. After washing 5 times with Tween-20/PBS, the plates were blocked with 5% horse serum/PBS (100 μl/well) for 1 hour, and then anti-MUC1 peptide antibodies BCP8 (anti-DTRPAPGST) or BCP9 (anti-GSTAPPAHG) (5 μg/ml in PBS 100 μl/well) (35) were added into the individual well for 2 h at RT. After washing with PBS, the plates were blocked with 1% horse serum/PBS (1000/well) for 1 hour. The plates were washed four times with PBS and incubated for 2 h at RT with horseradish-peroxidase-conjugated anti-mouse IgG (1:5000, Amersham, Piscataway, N.J.). Antibody complexes were detected by development with o-phenylenediamine (Sigma) and measured in a microplate autoreader EL310 (Bio-Rad, Hercules, Calif.) at an optical density of 492 nm.

Statistical analysis Statistical significance was analyzed using χ² and Student's t test. Percentage of positive cells in phenotype assay of T cells and DC was derived from two or three independent experiments and presented as means±SD.

### Example 1

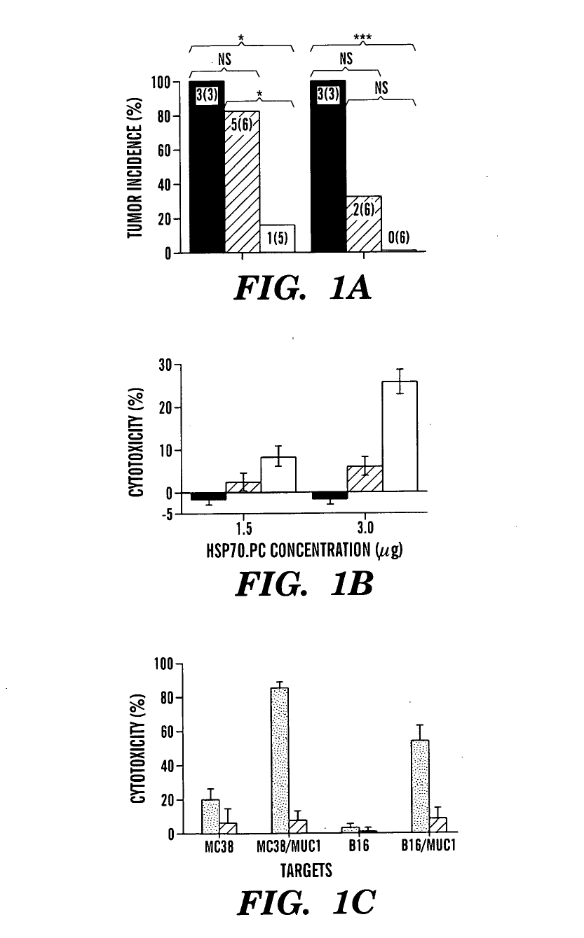

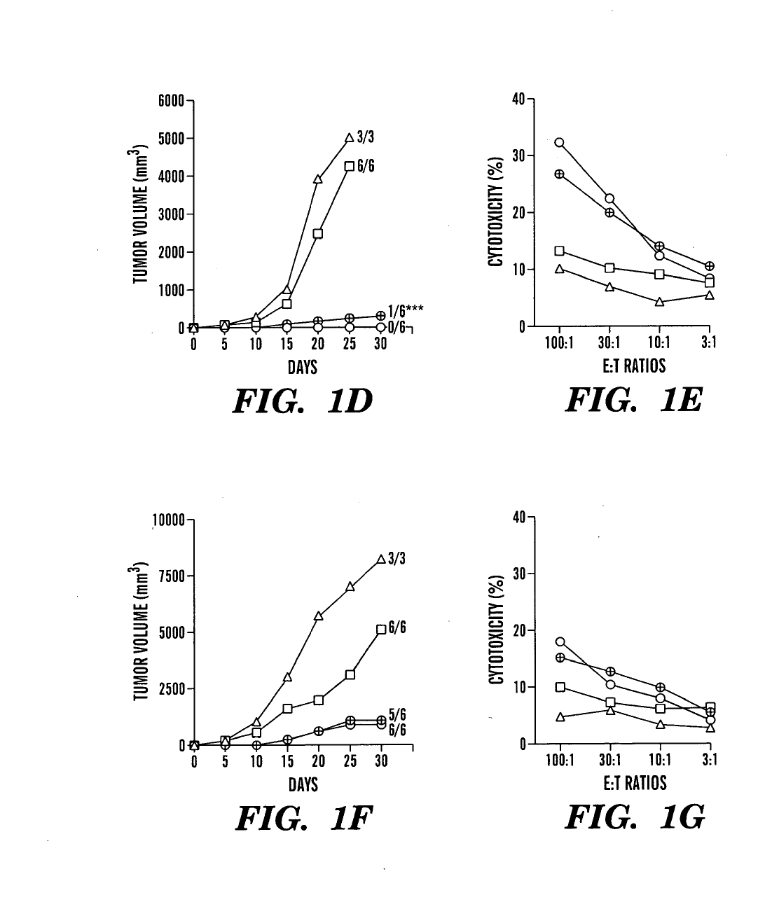

Enhanced antitumor immunity induced by HSP70-PC derived from fusion cells The inventors have examined the ability of HSP70 peptide complexes (HSP70-PC) from either tumor cells or DC-tumor fusion cells to immunize mice against tumor cells. HSP70-PC were immunoprecipitated from either fusion cells (HSP70.PC-F) or MC38/MUC1 tumor cells (HSP70.PC-Tu), using anti-HSP70 antibody Ab46 and the complexes were used as a tumor vaccine against MUC1 expressing MC38 cells (MC38/MUC1). Mice developed tumors in 100% of cases if untreated or sham treated with injection of buffer. Groups of wild-type mice were next vaccinated with HSP70.PC-F or HSP70.PC-Tu and then challenged with MC38/MUC1 tumor cells. Immunization with vaccine derived from tumor cells alone, at doses of 1.5 μg or 3.0 μg HSP70.PC-Tu resulted in partial protection and led to a reduction of tumor incidence to, respectively 83% at the lower dose (5 out of 6) and 33% (2 out of 6) at the higher dose of mice with tumor growth (FIG. 1A). By contrast, immunization with 3.0 μg of immunoprecipitate derived from the fusion cells protected all the mice from tumor challenge and only one mouse developed a tumor when immunized with the lower dose of 1.5 μg HSP70.PC-F (FIG. 1A). Extraction of HSP70-PC from DC fused to MC38/MUC1 tumor cells (FC/MUC1), therefore, increased efficacy in inhibiting tumor growth compared with mice immunized with HSP70.PC-Tu (P<0.05 at the dose of 1.5 μg). These anti-tumor responses correlated well with the results obtained in the CTL assay which showed that immunization of mice with HSP70.PC-F resulted in the highest CTL activity against these tumor cells that express MUC1 (FIG. 1B). Moreover, CTLs from WT mice immunized with HSP70.PC-F induces lysis of MC38/MUC1, B16/MUC1 and, to a lesser extent, MC38 cells, but not B16 cells (FIG. 1C). These results indicate that immunization with HSP70.PC-F induced immunity against MUC1 and other unknown antigens on MC38. Thus, the demonstration that B16/MUC1, and not B16, cells are lysed by the CTLs confirms that HSP70.PC-F induces MUC1-specific response. In addition, the lysis of MC38/MUC1, B16/MUC1 and MC38 cells was inhibited by anti-MHC class I mAb (FIG. 1C), suggesting MHC class I-restricted CTL activity. These experiments demonstrate that although both types of HSP70-based vaccine induce antitumor immunity, improved efficacy is observed in mice immunized with HSP70.PC-F, demonstrating enhanced immunogenicity of these preparations.

The inventors next tested the efficacy of HSP70.PC-F in MUC1 transgenic (MUC1.Tg) mice which have been engineered to over-express MUC1. Since the model antigen that was used in the experiments as disclosed herein is human MUC1, a tumor associated antigen expressed in approximately 72% of epithelial cancers (36), and it is possible that the observed immune response is derived from the reaction to human MUC1 as a xenoantigen in wild-type mice, an additional essential control was included.

The MUC1.Tg mouse has been shown to express MUC1 as a self-antigen (37), to develop tolerance in both B and T cell compartments and to be refractory to immunization with the MUC1 protein (25). The inventors used this model to determine whether HSP70 vaccines could break tolerance to non-mutated tumor antigens. MUC1.Tg mice were vaccinated twice with 1.5 μg HSP70-associated complexes derived from DC-tumor fusion cells or from tumor cells. Vaccination with HSP70-associated complexes derived from FC/MUC1 fusion cells generated strong CTL activity (FIG. 1E) and protected all but one mouse from challenge with tumor cells, an effect comparable to those vaccinated with DC-tumor fusion cells (FIG. 1D). By contrast, vaccination with the same dose of HSP70.PC-Tu generated low levels of CTL and failed to protect the mice from challenge with MUC1-positive tumor cells (FIGS. 1D and 1E). In control mice injected with PBS, tumors grew progressively. The preventive effect of the HSP70-FC vaccine is also statistically significant between mice immunized with HSP70.PC-F and those immunized with HSP70.PC-Tu (P<0.005). These results demonstrate that HSP70-PC derived from DC-tumor fusion vaccine are able to overcome the tolerance to a non-mutated tumor antigen and trigger immune response that provided protection of a host expressing MUC1 as a self antigen. This discovery has significant implication since most tumor antigens are non-mutated self antigens.

As HSP70 vaccines are commonly prepared using HSP70 isolated from tumors by ADP-affinity chromatography, the inventors examined this mode of HSP70 preparation also. The inventors obtained purified HSP70-peptide complexes from tumor and fusion cells using ADP-agarose affinity chromatography (38). Immunization of MUC1.Tg mice with such purified HSP70-peptide complexes from fusion cells (1.5 μg), although significantly reducing the rate of tumor growth resulted in suboptimal protection against tumor challenge although the growth rate was less than in mice immunized with purified HSP70-PC from tumor cells alone or PBS (FIG. 1F). Corresponding results were obtained using the CTL assay. Immunization with column purified HSP70-peptides induced moderate CTL activity against MC38/MUC1 (FIG. 1G). These results demonstrate the importance of isolation methodology in HSP70-PC preparation. The rationale behind these differences is not defined here but could include a number of mechanisms such as (a) the nature of the HSP70 isoforms isolated by the respective methods (b) relative association with/dissociation from MUC1 peptides and (c) the potential presence of other proteins co-precipitated with HSP70-PC. HSP70-PC obtained by rapid immunoprecipitation may preserve association with chaperoned peptides and other molecules, whereas HSP70-PC purification by sequential ATP agarose affinity chromatography, DEAE cellulose and Sephadex G25 chromatography (39), although highly effective in yielding a pure HSP70 preparation, may lead to dissociation of peptides and proteins especially considering the relatively low affinity of HSP70 polypeptide binding (Kd 10⁻⁶) and the prolonged nature of the isolation procedure (40).

Taken together, these results demonstrate that HSP70-PC preparations derived by immunoprecipitation from fusion cell lysates constitute an improved tumor vaccine. The immunogenicity of HSP70-PC from FC is significantly enhanced as determined by its ability to reverse T-cell tolerance to a self antigen and provide protection against challenge with tumor cells.

### Example 2

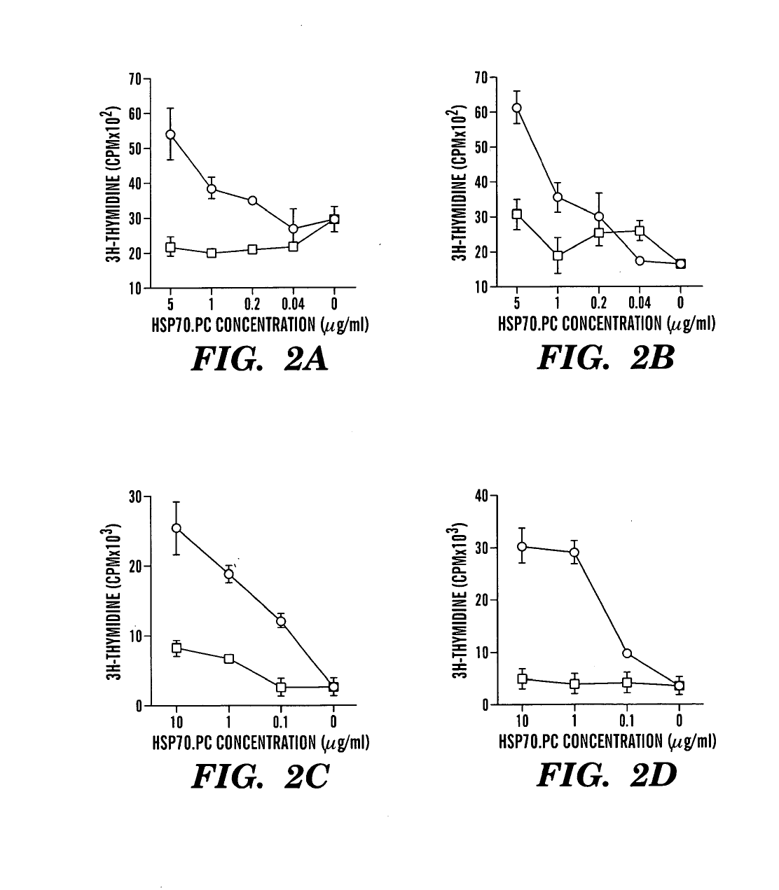

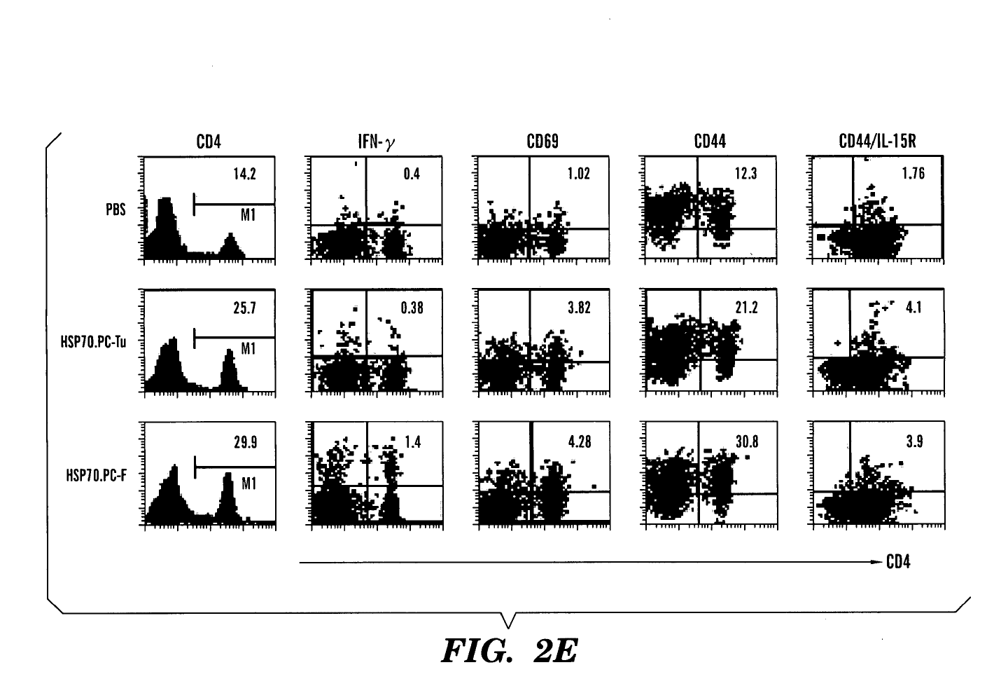

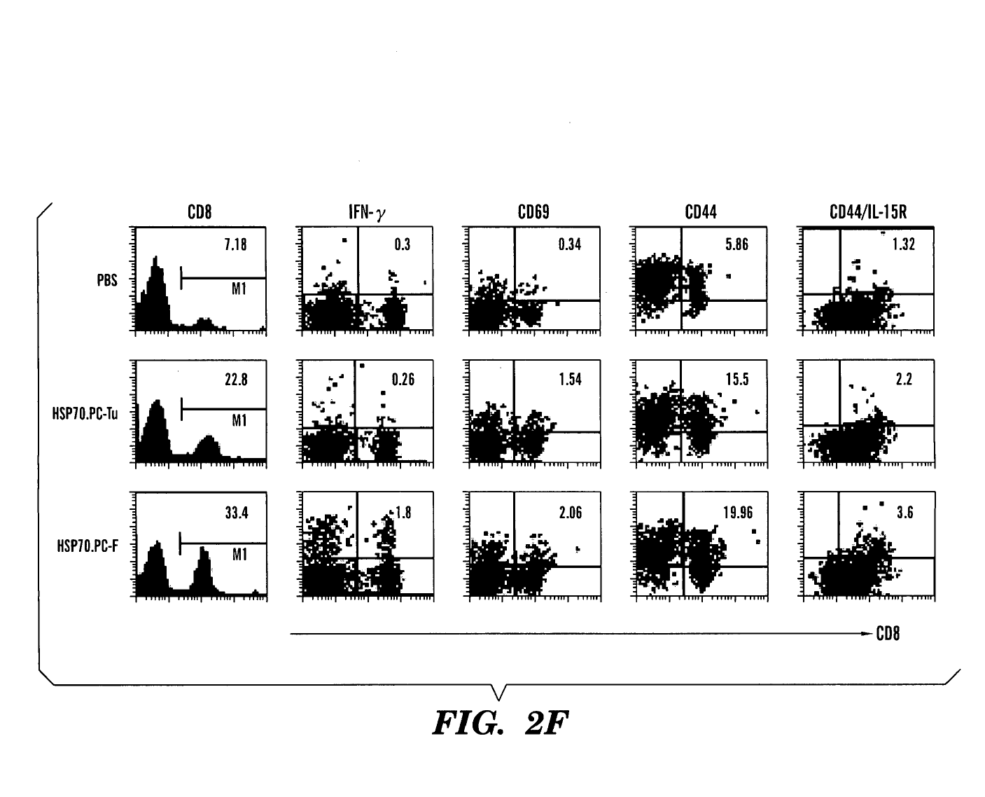

Enhanced stimulation of T cells by HSP70-PC from fusion cells The studies described in Example 1 demonstrate that HSP70.PC-F is superior to its counterpart from non-fused tumor cells in the induction of CLT and antitumor immunity. However, the data in Example I does not indicate the potential mechanisms underlying the increased immunogenicity. To determine if this enhanced anti-tumor immunity is achieved by a more robust induction and expansion of effector and memory T cells, the inventors assessed the ability of HSP70.PC-F to stimulate naïve T cells in vitro. The naïve lymph node cells (LNC) and splenocytes were isolated and co-cultured with HSP70.PC-F or HSP70.PC-Tu at indicated concentrations (FIGS. 2A and 2B). Naïve LNC and splenocytes were activated by the HSP70 preparations and proliferated more in the presence of HSP70.PC-F than those in the presence of HSP70.PC-Tu (FIGS. 2A and 2B). In addition, the CD4 and CD8 T cells from vaccinated MUC1.Tg mice were co-cultured with either HSP70.PC-F or HSP70.PC-Tu in the presence of DC. The CD4 and CD8 T cells proliferated when re-stimulated by HSP70.PC-F and, to a lesser extent, by HSP70.PC-Tu (FIGS. 2C and 2D). These experiments demonstrate the ability to stimulate T cells is enhanced with HSP70 from fusion cells.

To determine whether there are qualitative and quantitative differences in T cells primed by HSP70.PC-F and HSP70.PC-Tu in vivo, the inventors investigated the phenotypes of CD4 and CD8 T cells isolated from immunized mice. MUC1.Tg mice were immunized with HSP70-PC obtained from FC/MUC1 or tumor cells. LNC were then purified, stained with a panel of antibodies and quantitated by FACS analysis. Immunization with HSP70.PC-F, and to a lesser extent, with HSP70.PC-Tu resulted in increased numbers of CD4 and CD8 T cells in lymph nodes (LN) compared to the numbers of T cells in naïve mice (FIGS. 2E and F). A strikingly significant increase in CD8 T cell number was observed in mice immunized with HSP70.PC-F. Immunization with HSP70.PC-F resulted in 46% increase of CD8 T cells compared to mice immunized with HSP70.PC-Tu (FIGS. 2E and 2F). Importantly, CD4 or CD8 T cells expressing markers for activation, effector or memory T cells increased in the immunized mice (FIGS. 2E and 2F). The increase of CD4 and CD8 T cells expressing IFN-γ, CD69, CD44 and CD44/IL-15R was greatly enhanced in T cells isolated from mice immunized with hsp70.PC-F compared with mice immunized with HSP70.PC-Tu. The inventors discovered that HSP70.PC-F complexes possess a greatly enhanced capacity to stimulate T cells both in vivo and in vitro settings.

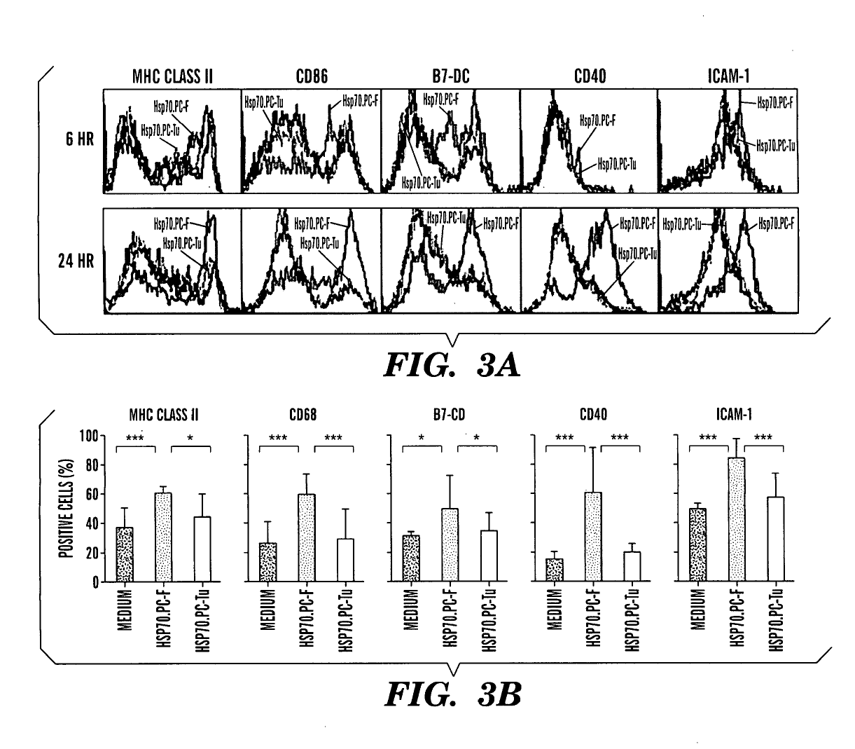

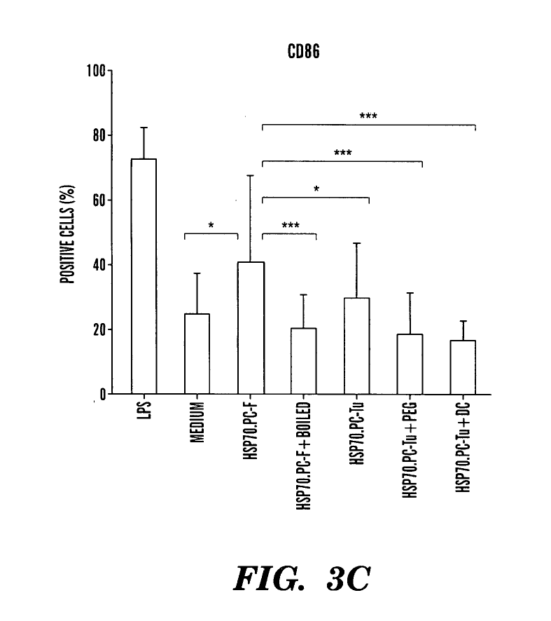

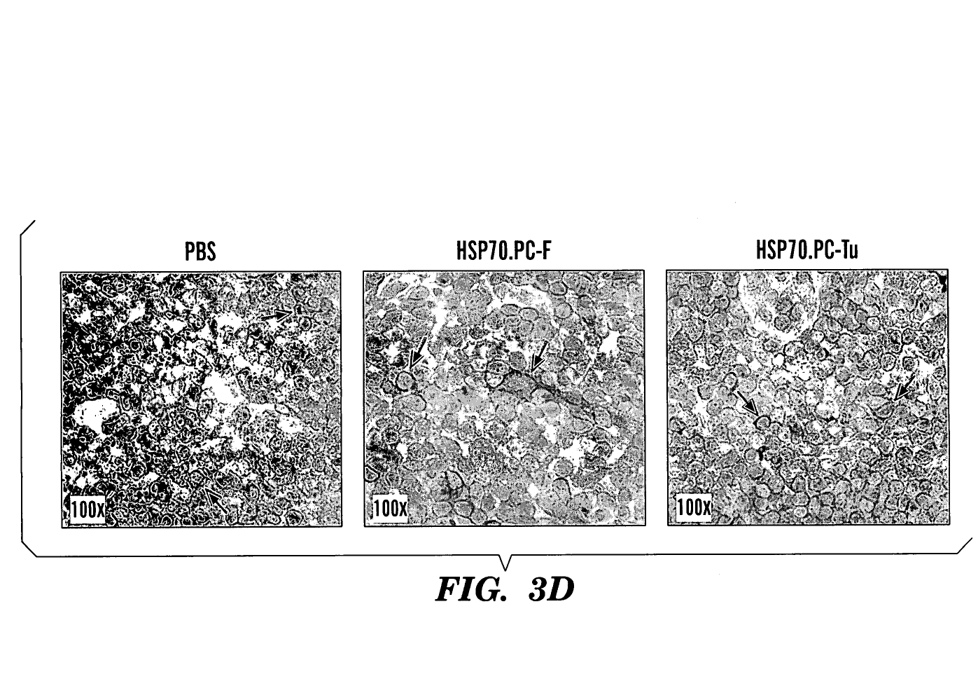

Enhanced maturation of DC by HSP70-PC from fusion cells. The inventors next determined whether T cell stimulation by HSP70-PC requires the participation of APC such as DC for antigen representation. DC maturation is essential for its ability to present antigen. The inventors assessed if HSP70.PC-F could stimulate DC to maturation. The inventors assessed the expression of MHC class II and co-stimulatory molecules on DC by generating immature DC and then co-cultured them with HSP70.PC-Tu, HSP70.PC-F or medium. Co-culture of DC with HSP70.PC-F resulted in significant up-regulation of MHC class II and co-stimulatory molecules in DC (FIGS. 3A and B). By contrast, up-regulation of these molecules in DC co-cultured with HSP70.PC-Tu was minimal. The inventors detected a statistical significant difference in DC maturation stimulated by HSP70.PC-F and HSP70.PC-Tu (FIG. 3B). As shown in FIGS. 3A and B, the up-regulation of MHC class II, CD86 and B7-DC was detected as early as 6 h after culture, while the up-regulation of CD40 and ICAM-1 was detected late in the culture.

To determine the role of DC-tumor fusion in the maturation of DC by HSP70, immature DC were cultured with HSP70-PC from various sources. The culture of immature DC with HSP70.PC-F resulted in up-regulation of CD86 molecules, although the level of increase is less than those stimulated by LPS (FIG. 3C). By contrast, comparable expression of CD86 was detected in control DC cultured with HSP70-PC derived from tumor cells, tumor-tumor fusion with Polyethylene glycol (PEG), mixture of DC and tumor cells or medium alone (FIG. 3C). Furthermore, the maturation effect of HSP70.PC-F was abrogated by boiling, determining a requirement for native tertiary structure in the effects of HSP70. The inventors detected a statistically significant difference in the up-regulation of CD86 molecules by HSP70.PC-F as compared to HSP70-PC derived from tumor cells, tumor-tumor fusion with Polyethylene glycol (PEG), mixture of DC and tumor cells or medium alone (FIG. 3C). These results demonstrate that physical fusion of DC and tumor cells prior to extraction of HSP70-PC is required for the increased maturation of DC by HSP70. In addition, it is unlikely that the maturation of DC by HSP70.PC-F is due to the contamination of endotoxin since the inventors used the same method and condition to prepare HSP70.PC-F and HSP70.PC-Tu and the inventors failed to observe the enhanced maturation of DC by HSP70.PC-Tu.

To assess the DC maturation in vivo by HSP70.PC-F, frozen sections were obtained from draining LN of mice immunized with HSP70-PC from tumor or fusion cells or injected with PBS, stained with mAb against CD86 and examined under microscopy. CD86-positive cells increased in mice vaccinated with HSP70.PC-F (data not shown). In contrast, few CD86-positive cells were observed in mice vaccinated with HSP70.PC-Tu or injected with PBS. Under high magnification, veiled cytoplasmic processes, a morphological characteristic of DC maturation, can be observed in CD86-positive cells from mice immunized with HSP70.PC-F (FIG. 3D). These results indicate that HSP70.PC from fusion cells possess enhanced stimulatory ability to mature DC compared to non-fused tumor cells.

### Example 3

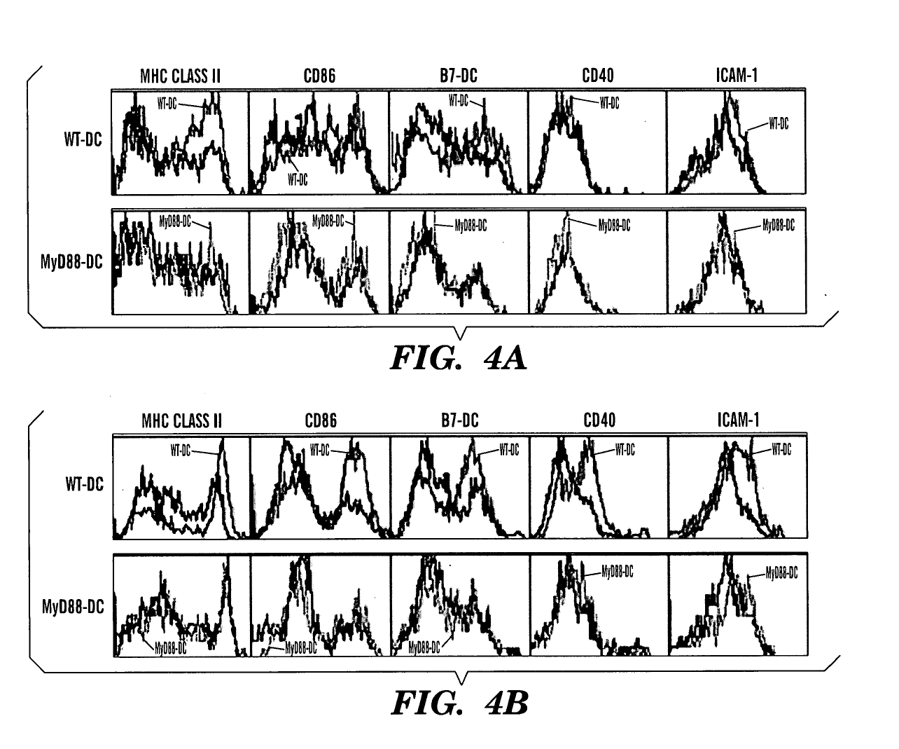

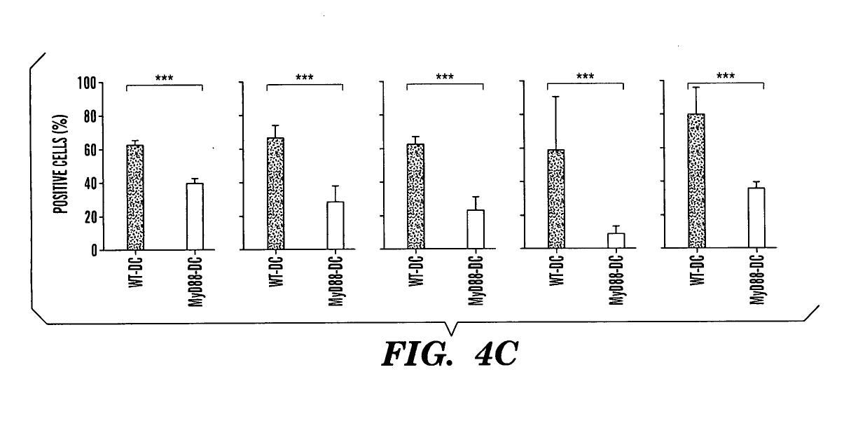

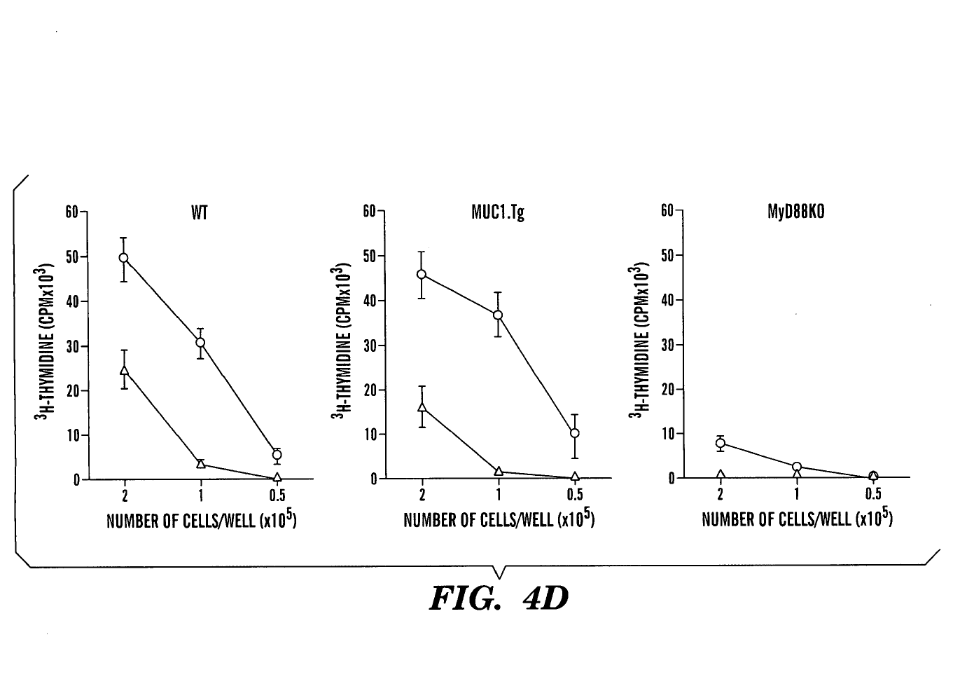

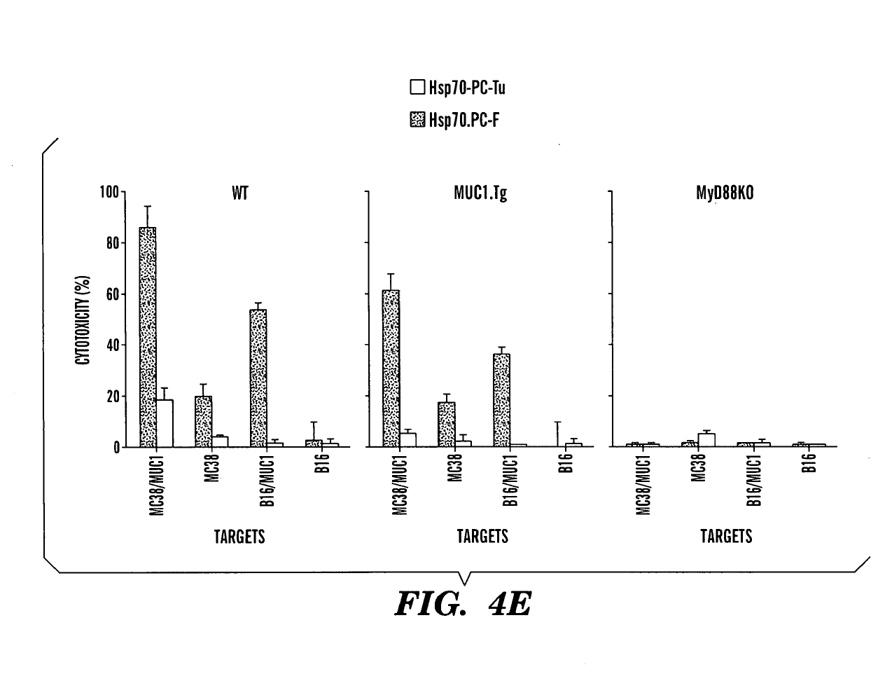

Involvement of MyD88 in the maturation of DC by HSP70-PC from fusion cells To study the potential signaling mechanism underlying such DC maturation, the inventors next cultured DC from mice deficient in MyD88^{-/-} or WT mice with HSP70.PC-F and compared their phenotype in response to the HSP70.PC-F. Stimulation of Toll-like receptors (TLR) plays a broad role in the maturation of DC (41) and MyD88 is an adaptor molecule required for downstream signaling for the TLR. Up-regulation of MHC class II and co-stimulatory molecules was observed on WT-DC co-cultured with HSP70.PC-F (FIG. 4A-C). In contrast, the effect of maturation of DC by HSP70.PC-F was obliterated in DC from MyD88 mice (FIG. 4A-C), resulting in comparable expression of MHC class II, CD86, B7-DC, CD40 or ICAM on DC from MyD88^{-/-} mice co-cultured with HSP70.PC-F to those co-cultured with medium at 6 hours (FIG. 4A) or 24 hours (FIG. 4B). To determine the involvement of MyD88 in vivo in the induction of T-cell mediated immunity, MyD88^{-/-}, MUC1.Tg and WT mice were immunized with HSP70.PC-F or HSP70.PC-Tu. Seven days after the second immunization, LNC and splenocytes were isolated and assayed for proliferation and CTL activity. T cells from immunized WT and MUC1.Tg mice proliferated vigorously (FIG. 4D). By contrast, minimal cell proliferation was observed in T cells from immunized MyD88^{-/-} mice (FIG. 4D). Similar results were obtained in CTL activity. CTLs from immunized WT and MUC1.Tg mice showed higher killings of tumor cells than those from immunized MyD88^{-/-} mice (FIG. 4E). In addition, the CTLs from WT and MUC1.Tg mice immunized with HSP70.PC-F lysed MC38/MUC1, B16/MUC1 and, to a lesser extent, MC38 tumor cells (FIG. 4E). By contrast, there was no, if any lysis of irrelevant B16 tumor cells. These results strongly demonstrate that the TLR/MyD88 pathway is involved in the maturation of DC by HSP70.PC-F and that enhanced innate immune stimulation by HSP70.PC-F may be responsible, at least in part, for the augmented antitumor immunity.

### Example 4

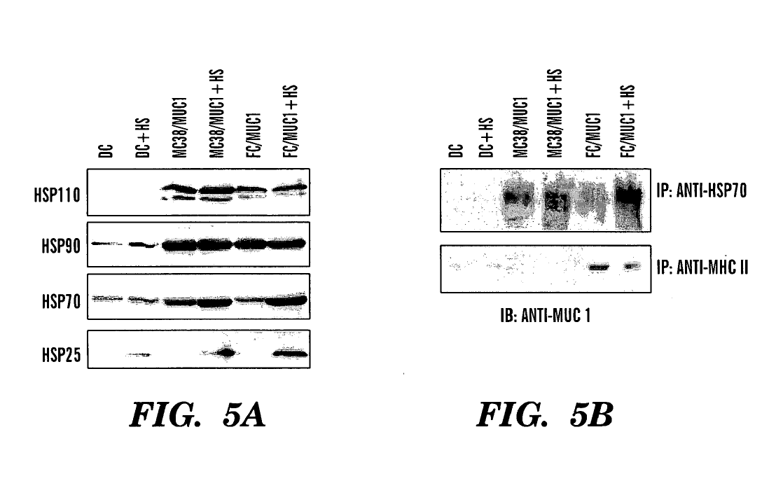

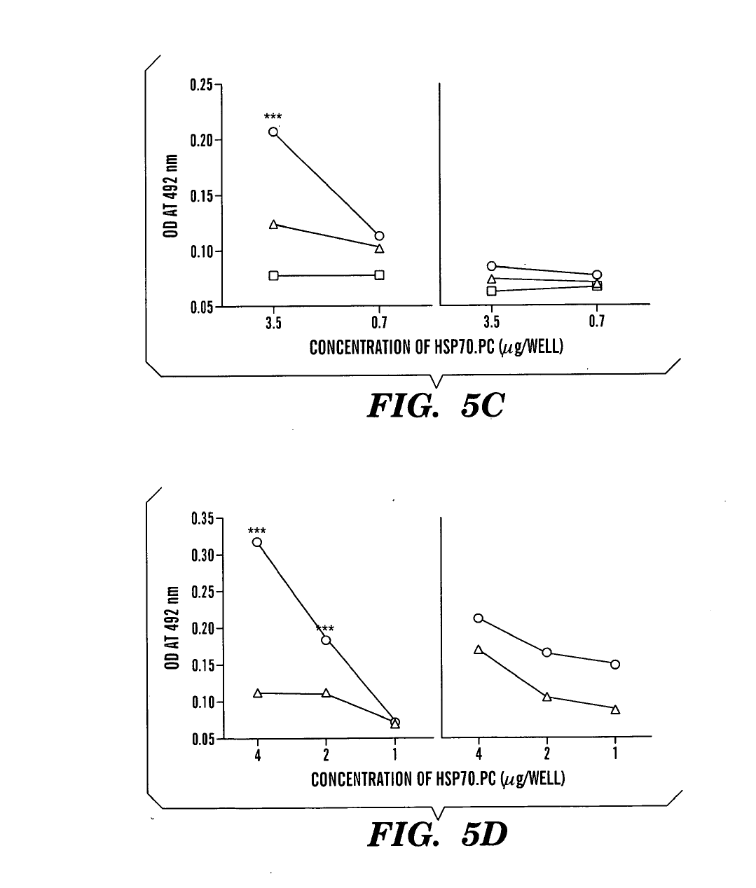

Enhanced MUC1 peptide association in HSP70 complexes from DC-tumor fusion cells. To determine the relative levels of HSP70 and other heat shock proteins in DC-tumor fusion cells and assess their association with tumor antigen MUC1, the inventors prepared lysates from heat shock-treated (HS) or untreated DC, MC38/MUC1 tumor cells and FC/MUC1 fusion cells. Extracts were loaded in equal concentrations into 6% or 10% SDS gel, transferred to nitrocellulose and immunoblotted with mAbs against HSP25, 70, 90 and 110. Although HSP expression in DC alone was relatively low, expression of HSP25 and HSP70 in FC/MUC1 fusion cells was increased by heat shock (FIG. 5A). To assess the association of HSP70 with MUC1, lysates from FC/MUC1 and MC38/MUC1 were immunoprecipitated with anti-HSP70 mAb followed by immunoblot analysis with anti-MUC1 mAb. Immunoprecipitation with anti-HSP70 antibody led to the co-precipitation of MUC1, indicating that HSP70 forms complexes with MUC1 (FIG. 5B), The relative density of MUC1 in lane 4 and 6 was 10.7% and 41.5%, respectively, suggesting stronger association of HSP70 with MUC1 in FC/MUC1 cells. The difference between lane 4 and 6 is statistical significance (P<0.005). These experiments indicate that DC-tumor fusion cells express heat shock proteins including HSP70 that are associated with tumor antigen.

To assess whether the isolated HSP70-PC carries immunogenic peptides, the inventors next measured the ability of HSP70-PC in association with antigenic peptides. The inventors obtained HSP70-PC using GST-HSP70 “pull-down” to optimize recovery of peptides. MC38/MUC1 carcinoma cells were transfected with a plasmid vector encoding the glutathione 5-transferase (GST) HSP70 fusion proteins or GST vector alone, and then fused to DC to create FC/MUC1/GSTHSP70 fusion cells or FC/MUC1/GST fusion cells, respectively. The wild-type GST and GSTHSP70 proteins were then isolated from those cells using GST pull-down. The inventors then assayed for the two individual MUC1 peptides; DTRPAPGST (SEQ ID NO: 1) and GSTAPPAHG (SEQ ID NO: 2) using ELISA with specific anti-peptide mAbs BCP8 and BCP9. MUC1 peptide DTRPAPGST (SEQ ID NO: 1) and GSTAPPAHG (SEQ ID NO: 2) were found to be restricted by H-2^{b} (35). Efficient association with BCP8 was observed in HSP70.PC-F while recovery was greatly reduced from MC38/MUC1-HSP70 tumor cells (FIG. 5C, left panel). In contrast, there was no reaction in complexes pulled down from vector-transfected tumor cells that express wild-type GST. The difference in MUC1 peptide content between HSP70.PC-F and HSP70.PC-Tu is statistical significant. Minimal association with BCP9 was observed in complexes pulled down from either cell types (FIG. 5C, right panel). To compare the level of peptide in HSP70-PC obtained by GST pull-down or ADP affinity chromatography, HSP70-PC from GST pull-down or ADP purification was assayed by BCP8 mAb. The recovery of MUC1 peptide was significantly reduced in HSP70.PC-F obtained by ADP affinity chromatography (FIG. 5D). Thus, the inventors have discovered HSP70-associated complexes carry tumor antigenic peptides and antigenic peptide association is increased in complexes from FC/MUC1. Thus, the inventors have discovered a method to increase the effectiveness of HSP.PC-F as a tumor vaccine. In addition, the inventors have discovered that the recovery of these peptides is affected by the method of extraction.

### Example 5

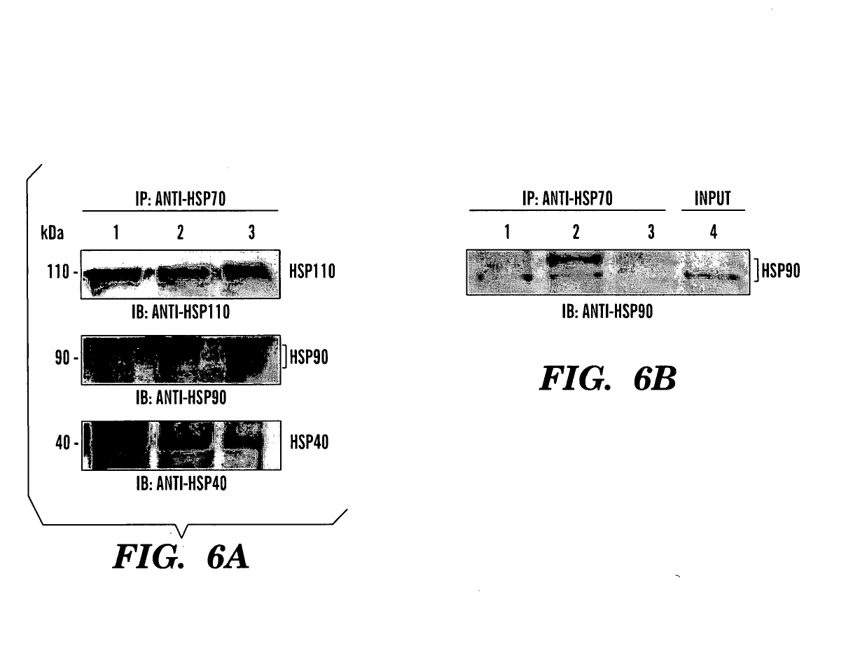

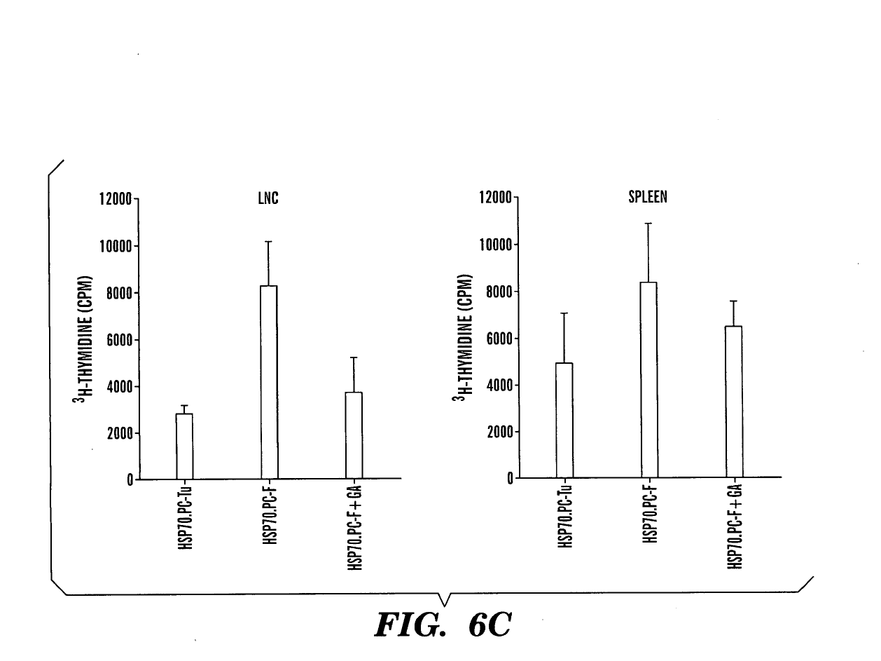

Promotion of association with HSP90 in HSP70 complexes from DC-tumor fusion cells. To determine whether HSP70 is associated with other proteins, lysates were next obtained from MC38/MUC1 or FC/MUC1 and immunoprecipitated with anti-HSP70 mAb. The immunoprecipitates were analyzed by immunoblotting with a panel of antibodies. FIG. 6A shows that HSP70 was associated with HSP90 and such association was strongly enhanced in HSP70.PC-F, whereas the association with HSP110 or HSP40 was comparable between HSP70.PC-F (lane 3) and HSP70.PC-Tu (lane 2). In addition, the enhanced association with HSP90 was not observed in the precipitates from the mixture of unfused DC and tumor cells (FIG. 6A, lane 1), demonstrating that physical fusion is essential for promotion of such association. To confirm the enhanced association with HSP90 in the precipitates from fusion cells, lysates were obtained from tumor cells, or FC/MUC1 treated with or without drug geldanamycin (GA) that is known to inhibit the HSP70-HSP90 interaction (42, 43). FIG. 6B shows that treatment of FC/MUC1 with geldanamycin abolished the association between HSP70 and HSP90 (lane 3). Interestingly, a novel HSP90 interacting band appeared in the HSP70 precipitates from FC/MUC1 (FIG. 6B, lane 2). This band is not observed in precipitates from FC/MUC1 treated with geldanamycin (lane 3) or tumor cells alone (lane 1), demonstrating that fusion of DC and tumor cells promotes the interaction with a novel HSP90 isoform. To assess the immunologic effect of HSP70 association with HSP90, naïve LNC and splenocytes were isolated and co-cultured with HSP70.PC-Tu, or HSP70.PC-F treated with or without geldanamycin. A higher rate of proliferation of LNC and splenocytes was detected in the culture stimulated by HSP70.PC-F (FIG. 6C). However, the stimulatory ability of HSP70.PC-F was diminished after pretreatment of FC/MUC1 with geldanamycin prior to HSP70 extraction (FIG. 6C). These results demonstrate that DC-tumor fusion promotes the association of HSP70 with HSP90 and that this HSP70-HSP90 complex enhances the immunogenicity of HSP70.PC-F.

As disclosed herein, the data clearly demonstrates a clear advantage for the molecular chaperone based vaccine extracted from DC-tumor fusion cells as compared to comparable extracts from tumor cells alone (FIGS. 1 and 2). The inventors discovered HSP70.PC-F can break T cell tolerance to a defined tumor antigen, kill MC38/MUC1 tumor cells and prevent tumor growth, as opposed to HSP70 preparations from tumor alone which were ineffective in breaking tolerance under the conditions used here. The inventors have discovered a method by which enhanced effects of HSP70.PC-F vaccine as disclosed herein can initiate the program of host DC maturation and lead to expression of co-stimulatory molecules required for T cell activation (FIG. 3). Host DC maturation initiated by the HSP70.PC-F vaccine leads to increases in CD4 and CD8 T cells expressing markers for activation, effector and memory T cells as seen in mice vaccinated with HSP70.PC-F vaccine.

The mechanisms underlying the effect of extracellular HSP70 on immune cells are only beginning to be determined (44). HSP70 has been shown to bind effectively to LOX-1, a member of the C-type lectin receptors which is found on the DC cell surface (45). Mechanism of signaling through LOX-1 have recently been shown to include the activation of Toll-like receptor 2 (TLR2), a potent activator of innate immunity (46). Indeed, the inventors have discovered herein that increased expression of markers of host DC maturation is abrogated by inactivation of the MyD88 gene, a key downstream component of TLR signaling (FIG. 4). A role for TLR signaling in stimulation of host APC by HSP70.PC-F is demonstrated as shown in FIG. 4. While the molecular mechanisms underlying the superiority of HSP70.PC-F as compared to HSP70 from tumor cells alone is complex, the inventors have demonstrated the HSP70.PC-F vaccine contains an elevated concentration of MUC1 antigenic peptides which can be cross-presented to T cells and lead to changes in T cell activation as observed in FIG. 2 and FIG. 5. The inventors have also discovered that antigenic peptides in addition to BCP8 as shown here can also be chaperoned by the HSP70.PC-F vaccine. The inventors can also use different species of HSP70 in the DC-tumor fusion cells. Human cells contain at least 12 HSP70 genes and the inventors have discovered that significant levels of 8 different HSP70 family gene products HSP70-1a, HSP70-1b, HSP70-HOM, HSP70-4, HSP70-5, HSP70-8, HSP70-9b, HSP75 and HSC70 are expressed in tumor cells (47, 48). Three of these (HSP70-1a, HSP70-1b, HSP70-HOM) are encoded by genes within the MHC locus suggesting immune function (49).

The previously described HSP70-PC based vaccines, although eliciting an immunological response, have been determined to be insufficient to provide protection against tumor growth (10, 13, 17). As disclosed herein, the inventors have discovered improvements to increase the efficacy of HSP70-PC as a therapeutic tumor vaccine. The inventors have discovered and demonstrated the following: (i) HSP70.PC-F possess superior adjuvant effect that matures the APC by up-regulation of MHC class II and co-stimulatory molecules over its counterpart from tumor cells, as shown in FIG. 3; (ii) HSP70.PC-F possess enhanced ability to induce effector and memory T cells, as shown in FIG. 2; (iii) vaccination of mice with HSP70.PC-F confers sufficient protection against the challenge with tumor cells, as shown in FIG. 1; (iv) HSP70.PC-F can break T-cell tolerance to a predominant tumor antigen MUC1, as shown in FIG. 1); (v) HSP70-PC derived from DC-tumor fusion vaccine are strongly associated with tumor antigen and contains enriched antigenic peptides, as shown in FIG. 5 (FIG. 5); (vi) HSP70 from fusion cells associates with HSP90, as shown in FIG. 6, which demonstrates its ability to carry antigenic peptides (50). Therefore, the inventors have demonstrated a method to significant improve the advancement of cancer immunotherapy.

The molecular events that promote the formation of immunogenic complexes of HSP with tumor antigenic peptides in fusion cell are unclear. Previous studies show that fusion of DC and tumor cell creates an immunogenic cell with integration of cytoplasm of these two cells (51). Such integration makes it possible to exchange cytoplasmic contents and share subcellular compartments between DC and tumor cell. Indeed, the inventors have demonstrated the expression of tumor antigen on the surface of the DC side and DC marker on the surface of tumor cell side of the fusion cells (51). The inventors have discovered a method to obtain HSP-based vaccines from a fusion cell which is a combination of DC and tumor cell. It has been recognized that DC are the most potent APC in the body possessing efficient antigen processing (endosome/lysosome) and presentation machinery, whereas tumor cells express abundant tumor antigens. The inventors have discovered the fusion cells inherit the antigen-processing and presentation machinery from DC and possess the full set of accessory molecules necessary for antigen processing and presentation. These molecules facilitate or participate in the antigen processing and presentation of tumor antigens, thus the inventors have discovered a method for antigen processing and presentation of tumor antigens enriched in a wider repertoire of immunogenic peptides. In addition, the inventors have discovered that the fusion cells inherit from tumor cells the ability to synthesize tumor antigen de novo, thus making these synthesized tumor antigens accessible to endogenous processing pathway. The inventors have also demonstrated that the antigen-processing machinery from DC can sort or select the immunogenic peptide to be processed and presented and work much more efficiently than those from tumor cells, thus increasing the quality and quantity of the HSP-associated complexes.

[The inventors have also discovered the induction of a robust CD4 and CD8 T cell response by HSP70-PC derived from fusion cells contributes to the efficacy of HSP70.PC-F. The inventors demonstrate that immunization with HSP70.PC-F and, to a less extent, with HSP70.PC-Tu resulted in the significant proliferation of CD4 and CD8 T cells and increased the T cell populations expressing effector and memory markers. The induction of these T cells by HSP70.PC-F was demonstrated to contribute to the enhanced antitumor immunity, and protected the immunized mice. It should be noted that the populations of fusion cells expressing CD69, CD44, CD44/IL-15R and IFN-γ also increased in CD4 T cells from HSP70.PC-F-immunized mice. There is increasing evidence that CD4 T cells activated by MHC class II-restricted epitope play a critical role in the antitumor immunity (52, 53).

In the present study, the inventors have largely concentrated on HSP70 since HSP70 chaperone proteins are associated with the immunogenicity of DC-tumor fusion cells (unpublished data). However, other stress proteins, with markedly different peptide binding domains bind to different spectra of cellular peptide antigens can also be used in the methods as disclosed herein for antitumor immunity (54, 55).

### Example 6

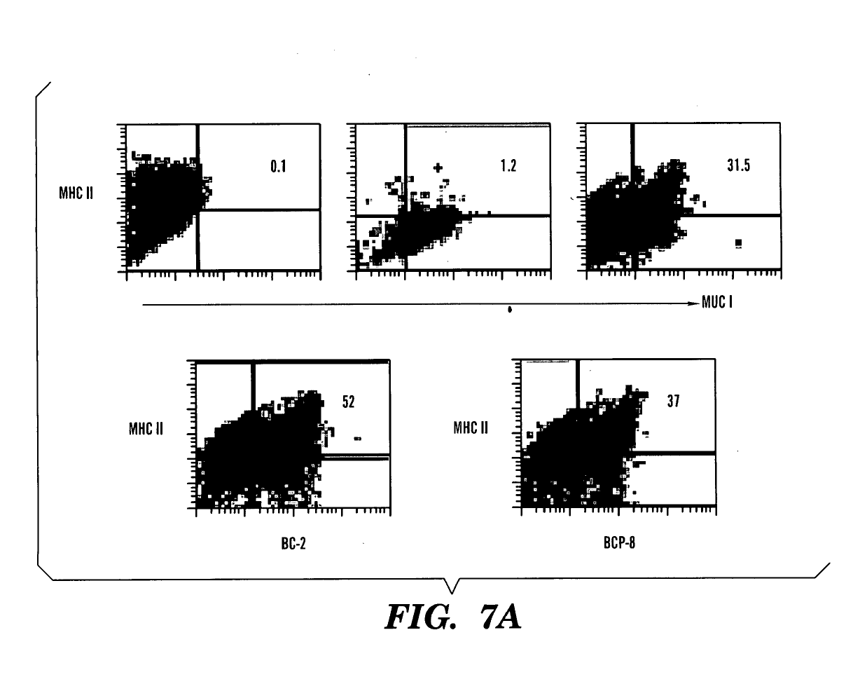

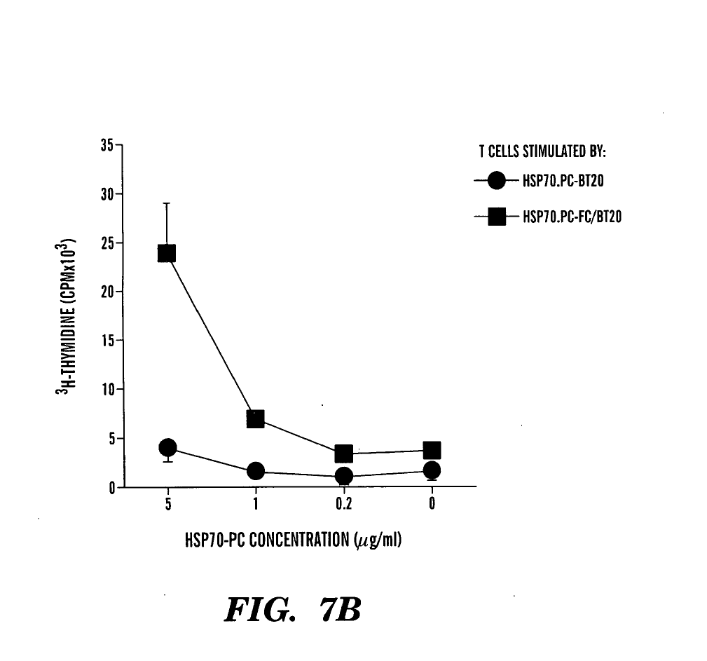

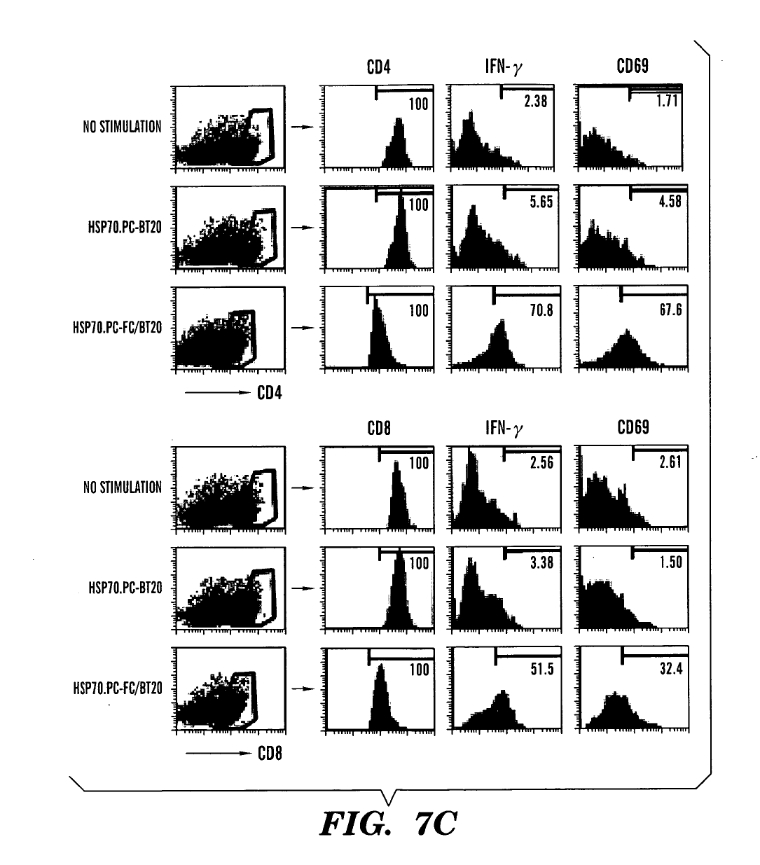

Enhanced immunogenecity of HSP70 complexes obtained from human fusion cells. To develop a molecular chaperone based vaccine with enhanced immunogenicity for human use, for example for human patients, the inventors have extracted HSP70-PC carrying immunogenic peptides from shared tumor antigens such as MUC1 and Her2/neu from human DC fused to established tumor cells (FC). DC were generated from peripheral blood mononuclear cells (PBMC) from healthy donors and fused to breast cancer cells BT20 in the presence of 50% PEG as described previously (56). FACS analysis shows that DCs were positive for MHC class II, whereas BT20 were positive for MUC1 as well as MUC1 peptide (FIG. 7A). In contrast, fusions of DC and BT20 were stained positive for MHC class II and MUC1 or MUC1 peptide (BCP-8). These results demonstrate that the fusion cells possess the phenotypic properties of their parent cells and that tumor antigens, such as MUC1, is processed by the fusion cells.

The inventors next used immunoprecipitation method as previously described (57) to obtain HSP70 complexes from fusions of DC and BT20 tumor cells (HSP70.PC-FC/BT20) or tumor cells alone (HSP70.PC-BT20). To determine the immunogenecity of the HSP70 complexes extracted from fusion cells, naïve PBMC positive for HLA-A*02 from a donor were co-cultured with HSP70-PC extracted from tumor or fusion cells, respectively. Co-culture of PBMC with HSP70-PC from fusion cells resulted in the proliferation of T cells, as shown in FIG. 7B. By contrast, minimal T cell proliferation was observed when co-cultured with HSP70-PC extracted from tumor cells. In addition, increased number of CD4 and CD8 T cells expressing IFN-γ and CD69 were stimulated by HSP70.PC-FC/BT20 and, to a lesser extent, by HSP70.PC-BT20 (FIG. 7C). Similar results were obtained in the HSP70-PC extracted from DC fused to MCF7 breast cancer cells (data not shown). Take together, these results demonstrate that HSP70-PC extracted from human DC-tumor fusion cells possess enhanced immunogenicity.

### Example 7

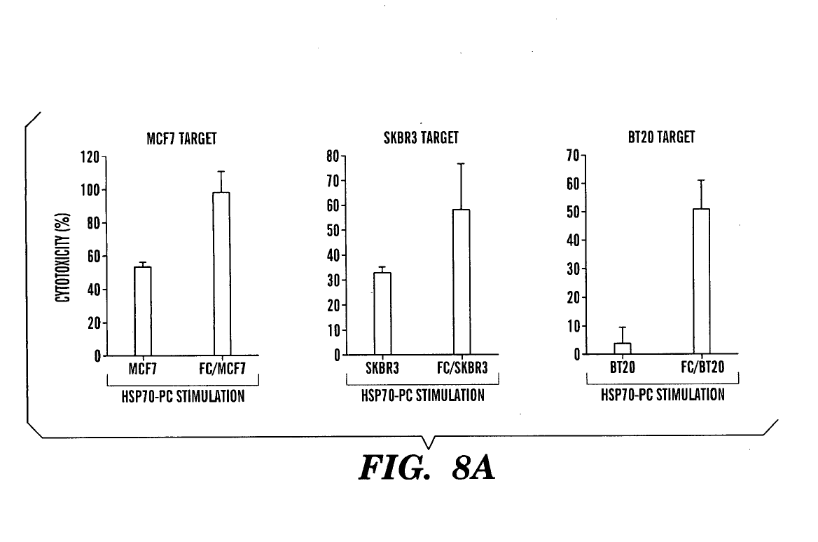

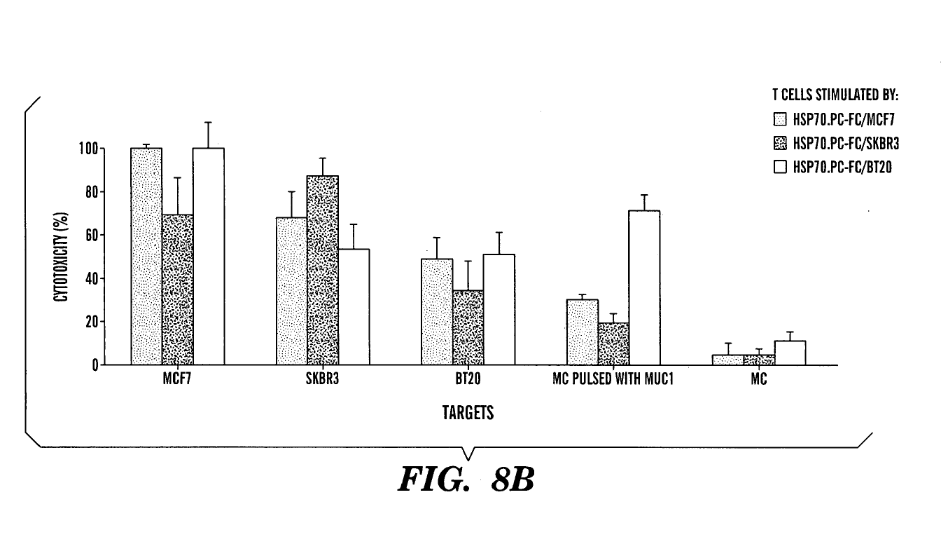

To compare the CTL activity induced by HSP70-PC extracted from DC-tumor fusion cells or tumor cells alone, the inventors fused DC to various breast cancer cells. Fusion of DC with MCF7, SKBR3 or BT20 breast cancer cells resulted in FC/MCF7, FC/SKBR3 or FC/BT20, respectively. HSP70-PC were extracted from FC/MCF7, FC/SKBR3 or FC/BT20 using immunoprecipitation as described (57), resulting in HSP70.PC-FC/MCF7, HSP70.PC-FC/SKBR3 and HSP70.PC-FC/BT20. HSP70-PC immunoprecipited from MCF7, SKBR3 or BT20 breast cancer cells were used as controls. These complexes were used to induce CTL activity. Naïve PBMC positive for HLA-A*02 and HLA-A*11 from a donor were co-cultured with HSP70.PC-FC/MCF7, HSP70.PC-FC/SKBR3, HSP70.PC-FC/BT20 and control HSP70-PC from MCF7, SKBR3 or BT20 breast cancer cells, respectively. Higher level of CTL activity induced by HSP70-PC from fusion cells against breast cancer cells was observed than those from their counterparts from cancer cells (FIG. 8A). After co-cultured with HSP70.PC-FC/MCF7, HSP70.PC-FC/SKBR3 or HSP70.PC-FC/BT20, the T cells were functional in lysis of MCF7, SKBR3 and BT-20, respectively. To determine the specificity of the CTL, T cells induced by HSP70.PC-FC/MCF7, HSP70.PC-FC/SKBR3 or HSP70.PC-FC/BT20 were incubated with multiple tumor targets. T cells induced by HSP70.PC-FC/MCF7, HSP70.PC-FC/SKBR3 or HSP70.PC-FC/BT20 were killed not only MCF7, but also the other MUC1 or Her-2/neu-positive breast caner cells, demonstrating that polyclonal CTL was induced by HSP70-PC extracted from DC-tumor fusion cells, as shown in FIG. 8B. In addition, these T cells also lysed monocytes pulsed with MUC1 but not monocytes. These results demonstrate tumor antigen-specificity of CTL induced by HSP70-PC from fusion cells.

### REFERENCES

All references cited herein are incorporated herein by reference in their entirety as if each individual publication or patent or patent application was specifically and individually indicated to be incorporated by reference in its entirety.

Many modifications and variations of this invention can be made without departing from its spirit and scope, as will be apparent to those skilled in the art. The specific embodiments described herein are offered by way of example only, and the invention is to be limited only in terms of the appended claims, along with the full scope of equivalents to which such claims are entitled.
1. Lindquist, S., and E. A. Craig. 1988. The heat-shock proteins. Annu. Rev. Genet. 22: 631-677.
2. Georgopoulos, C., and W. J. Welch. 1993. Role of the major heat shock proteins as molecular chaperones. Annu. Rev. Cell Biol. 9: 601-634.
3. Udono, H., and P. K. Srivastava. 1993. Heat shock protein 70-associated peptides elicit specific cancer immunity. J. Exp. Med. 178: 1391-1396.
4. Arnold, D., S. Faath, H. Rammensee, and H. Schild. 1995. Cross-priming of minor histocompatibility antigen-specific cytotoxic T cells upon immunization with the heat shock protein gp96. J. Exp. Med. 182: 885-889.
5. Heikema, A., E. Agsteribbe, J. Wilschut, and A. Huckriede. 1997. Generation of heat shock protein-based vaccines by intracellular loading of gp96 with antigenic peptides. Immunol. Lett. 57: 69-74.
6. Ciupitu, A. M., M. Petersson, C. L. O'Donnell, K. Williams, S. Jindal, R. Kiessling, and R. M. Welsh. 1998. Immunization with a lymphocytic choriomeningitis virus peptide mixed with heat shock protein 70 results in protective antiviral immunity and specific cytotoxic T lymphocytes. J. Exp. Med. 187: 685-691.
7. Suzue, K., and R. A. Young. 1996. Adjuvant-free hsp70 fusion protein system elicits humoral and cellular immune responses to HIV-1 p24. J. Immunol. 156: 873-879.
8. Chen, C. H., T. L. Wang, C. F. Hung, Y. Yang, R. A. Young, D. M. Pardoll, and T. C. Wu. 2000. Enhancement of DNA vaccine potency by linkage of antigen gene to an HSP70 gene. Cancer Res. 60: 1035-1042.
9. Cho, B. K., D. Palliser, E. Guillen, J. Wisniewski, R. A. Young, J. Chen, and H. N. Eisen. 2000. A proposed mechanism for the induction of cytotoxic T lymphocyte production by heat shock fusion proteins. Immunity 12: 263-272.
10. Moroi, Y., M. Mayhew, J. Trcka, M. H. Hoe, Y. Takechi, F. U. Hartl, J. E. Rothman, and A. N. Houghton. 2000. Induction of cellular immunity by immunization with novel hybrid peptides complexed to heat shock protein 70. Proc. Natl. Acad. Sci. USA 97: 3485-3490.
11. Castelli, C., A. M. Ciupitu, F. Rini, L. Rivoltini, A. Mazzocchi, R. Kiessling, and G. Parmiani. 2001. Human heat shock protein 70 peptide complexes specifically activate antimelanoma T cells. Cancer Res. 61: 222-227.
12. Binder, R. J., and P. K. Srivastava. 2005. Peptides chaperoned by heat-shock proteins are a necessary and sufficient source of antigen in the cross-priming of CD8+ T cells. Nat. Immunol. 6: 593-599.
13. Huang, C., H. Yu, Q. Wang, W. Ma, D. Xia, P. Yi, L. Zhang, and X. Cao. 2004. Potent antitumor effect elicited by superantigen-linked tumor cells transduced with heat shock protein 70 gene. Cancer Sci. 95: 160-167.
14. Srivastava, P. K., A. B. DeLeo, and L. J. Old. 1986. Tumor rejection antigens of chemically induced sarcomas of inbred mice. Proc. Natl. Acad. Sci. USA 83: 3407-3411.
15. Udono, H., D. L. Levey, and P. K. Srivastava. 1994. Cellular requirements for tumor-specific immunity elicited by heat shock proteins: tumor rejection antigen gp96 primes CD8+ T cells in vivo. Proc. Natl. Acad. Sci. USA 91: 3077-3081.
16. Tamura, Y., P. Peng, K. Liu, M. Daou, and P. K. Srivastava. 1997. Immunotherapy of tumors with autologous tumor-derived heat shock protein preparations. Science 278: 117-120.
17. Vanaja, D. K., M. E. Grossmann, E. Celis, and C. Y. Young. 2000. Tumor prevention and antitumor immunity with heat shock protein 70 induced by 15-deoxy-delta12,14-prostaglandin J2 in transgenic adenocarcinoma of mouse prostate cells. Cancer Res. 60: 4714-4718.
18. Nicchitta, C. V. 1998. Biochemical, cell biological and immunological issues surrounding the endoplasmic reticulum chaperone GRP94/gp96. Curr. Opin. Immunol. 10: 103-109.
19. Janetzki, S., D. Palla, V. Rosenhauer, H. Lochs, J. J. Lewis, and P. K. Srivastava. 2000. Immunization of cancer patients with autologous cancer-derived heat shock protein gp96 preparations: a pilot study. Int. J. Cancer 88: 232-238.
20. Heike, M., A. Weinmann, K. Bethke, and P. R. Galle. 1999. Stress protein/peptide complexes derived from autologous tumor tissue as tumor vaccines. Biochem. Pharmacol. 58: 1381-1387.
21. Przepiorka, D., and P. K. Srivastava. 1998. Heat shock protein—peptide complexes as immunotherapy for human cancer. Mol. Med. Today 4: 478-484.
22. Belli, F., A. Testori, L. Rivoltini, M. Maio, G. Andreola, M. R. Sertoli, G. Gallino, A. Piris, A. Cattelan, I. Lazzari, M. Carrabba, G. Scita, C. Santantonio, L. Pilla, G. Tragni, C. Lombardo, F. Arienti, A. Marchiano, P. Queirolo, F. Bertolini, A. Cova, E. Lamaj, L. Ascani, R. Camerini, M. Corsi, N. Cascinelli, J. J. Lewis, P. Srivastava, and G. Parmiani. 2002. Vaccination of metastatic melanoma patients with autologous tumor-derived heat shock protein gp96-peptide complexes: clinical and immunologic findings. J. Clin. Oncol. 20: 4169-4180.
23. Mazzaferro, V., J. Coppa, M. G. Carrabba, L. Rivoltini, M. Schiavo, E. Regalia, L. Mariani, T. Camerini, A. Marchiano, S. Andreola, R. Camerini, M. Corsi, J. J. Lewis, P. K. Srivastava, and G. Parmiani. 2003. Vaccination with autologous tumor-derived heat-shock protein gp96 after liver resection for metastatic colorectal cancer. Clin. Cancer Res. 9: 3235-3245.
24. Parmiani, G., A. Testori, M. Maio, C. Castelli, L. Rivoltini, L. Pilla, F. Belli, V. Mazzaferro, J. Coppa, R. Patuzzo, M. R. Sertoli, A. Hoos, P. K. Srivastava, and M. Santinami. 2004. Heat shock proteins and their use as anticancer vaccines. Clin. Cancer Res. 10: 8142-8146.
25. Rowse, G. J., R. M. Tempero, M. L. VanLith, M. A. Hollingsworth, and S. J. Gendler. 1998. Tolerance and immunity to MUC1 in a human MUC1 transgenic murine model. Cancer Res. 58: 315-321.
26. Siddiqui, J., M. Abe, D. Hayes, E. Shani, E. Yunis, and D. Kufe. 1988. Isolation and sequencing of a cDNA coding for the human DF3 breast carcinoma-associated antigen. Proc. Natl. Acad. Sci. USA 85: 2320-2323.
27. Akagi, J., J. W. Hodge, J. P. McLaughlin, L. Gritz, G. Mazzara, D. Kufe, J. Schlom, and J. A. Kantor. 1997. Therapeutic antitumor response after immunization with an admixture of recombinant vaccinia viruses expressing a modified MUC1 gene and the murine T-cell costimulatory molecule B7. J. Immunother. 20: 38-47.
28. Inaba, K., M. Inaba, N. Romani, H. Aya, M. Deguchi, S. Ikehara, S. Muramatsu, and R. M. Steinman. 1992. Generation of large numbers of dendritic cells from mouse bone marrow cultures supplemented with granulocyte/macrophage colony-stimulating factor. J. Exp. Med. 176: 1693-1702.
29. Gong, J., D. Chen, M. Kashiwaba, and D. Kufe. 1997. Induction of antitumor activity by immunization with fusions of dendritic and carcinoma cells. Nat. Med. 3: 558-561.
30. Tanaka, Y., S. Koido, M. Ohana, C. Liu, and J. Gong. 2005. Induction of Impaired Antitumor Immunity by Fusion of MHC Class II-Deficient Dendritic Cells with Tumor Cells. J. Immunol. 174: 1274-1280.
31. Koido, S., Y. Tanaka, D. Chen, D. Kufe, and J. Gong. 2002. The kinetics of in vivo priming of CD4 and CD8 T cells by dendritic/tumor fusion cells in MUC1-transgenic mice. J. Immunol. 168: 2111-2117.
32. Stevenson, M. A., and S. K. Calderwood. 1990. Members of the 70-kilodalton heat shock protein family contain a highly conserved calmodulin-binding domain. Mol. Cell. Biol. 10: 1234-1238.
33. Gong, J., V. Apostolopoulos, D. Chen, H. Chen, S. Koido, S. J. Gendler, I. F. McKenzie, and D. Kufe. 2000. Selection and characterization of MUC1-specific CD8+ T cells from MUC1 transgenic mice immunized with dendritic-carcinoma fusion cells. Immunology 101: 316-324.
34. Kantor, J., K. Irvine, S. Abrams, H. Kaufman, J. DiPietro, and J. Schlom. 1992. Antitumor activity and immune responses induced by a recombinant carcinoembryonic antigen-vaccinia virus vaccine. J. Natl. Cancer Inst. 84: 1084-1091.
35. Apostolopoulos, V., G. Chelvanayagam, P. X. Xing, and I. F. McKenzie. 1998. Anti-MUC1 antibodies react directly with MUC1 peptides presented by class I H2 and HLA molecules. J. Immunol. 161: 767-775.
36. Greenlee, R. T., T. Murray, S. Bolden, and P. A. Wingo. 2000. Cancer statistics, 2000. CA Cancer J. Clin. 50: 7-33.
37. Peat, N., S. J. Gendler, N. Lalani, T. Duhig, and J. Taylor-Papadimitriou. 1992. Tissue-specific expression of a human polymorphic epithelial MUC1n (MUC1) in transgenic mice. Cancer Res. 52: 1954-1960.
38. Peng, P., A. Menoret, and P. K. Srivastava. 1997. Purification of immunogenic heat shock protein 70-peptide complexes by ADP-affinity chromatography. J. Immunol. Methods 204: 13-21.
39. Asea, A., E. Kabingu, M. A. Stevenson, and S. K. Calderwood. 2000. HSP70 peptide-bearing preparations act as chaperokines. Cell Stress Chaperones 5: 425-431.
40. Calderwood, S. K., J. R. Theriault, and J. Gong. 2005. Message in a bottle: role of the 70-kDa heat shock protein family in anti-tumor immunity. Eur. J. Immunol. 35: 2518-2527.
41. Liu, B., J. Dai, H. Zheng, D. Stoilova, S. Sun, and Z. Li. 2003. Cell surface expression of an endoplasmic reticulum resident heat shock protein gp96 triggers MyD88-dependent systemic autoimmune diseases. Proc. Natl. Acad. Sci. USA 100: 15824-15829.
42. Mayer, M. P., and B. Bukau. 2005. Hsp70 chaperones: cellular functions and molecular mechanism. Cell Mol. Life. Sci. 62: 670-684.
43. Roe, S. M., C. Prodromou, R. O'Brien, J. E. Ladbury, P. W. Piper, and L. H. Pearl. 1999. Structural basis for inhibition of the Hsp90 molecular chaperone by the antitumor antibiotics radicicol and geldanamycin. J. Med. Chem. 42: 260-266.
44. Calderwood, S. K., J. R. Theriault, and J. Gong. 2005. How is the immune response affected by hyperthermia and heat shock proteins? Int. J. Hyperthermia 21: 713-716.
45. Delneste, Y., G. Magistrelli, J. Gauchat, J. Haeuw, J. Aubry, K. Nakamura, N. Kawakami-Honda, L. Goetsch, T. Sawamura, J. Bonnefoy, and P. Jeannin. 2002. Involvement of LOX-1 in dendritic cell-mediated antigen cross-presentation. Immunity 17: 353-362.
46. Jeannin, P., B. Bottazzi, M. Sironi, A. Doni, M. Rusnati, M. Presta, V. Maina, G. Magistrelli, J. F. Haeuw, G. Hoeffel, N. Thieblemont, N. Corvaia, C. Garlanda, Y. Delneste, and A. Mantovani. 2005. Complexity and complementarity of outer membrane protein A recognition by cellular and humoral innate immunity receptors. Immunity 22: 551-560.
47. Bukau, B., and A. L. Horwich. 1998. The Hsp70 and Hsp60 chaperone machines. Cell 92: 351-366.
48. Tang, D., M. A. Khaleque, E. L. Jones, J. R. Theriault, C. Li, W. H. Wong, M. A. Stevenson, and S. K. Calderwood. 2005. Expression of heat shock proteins and heat shock protein messenger ribonucleic acid in human prostate carcinoma in vitro and in tumors in vivo. Cell Stress Chaperones 10: 46-58.
49. Fourie, A. M., P. A. Peterson, and Y. Yang. 2001. Characterization and regulation of the major histocompatibility complex-encoded proteins Hsp70-Hom and Hsp70-1/2. Cell Stress Chaperones 6: 282-295.
50. Ren, J., A. Bharti, D. Raina, W. Chen, R. Ahmad, and D. Kufe. 2006. MUC1 oncoprotein is targeted to mitochondria by heregulin-induced activation of c-Src and the molecular chaperone HSP90. Oncogene 25: 20-31.
51. Koido, S., M. Ohana, C. Liu, N. Nikrui, J. Durfee, A. Lerner, and J. Gong. 2004. Dendritic cells fused with human cancer cells: morphology, antigen expression, and T cell stimulation. Clin. Immunol. 113: 261-269.
52. Ostrand-Rosenberg, S. 2005. CD4+ T lymphocytes: a critical component of antitumor immunity. Cancer Invest. 23: 413-419.
53. Toes, R. E., F. Ossendorp, R. Offring a, and C. J. Melief. 1999. CD4 T cells and their role in antitumor immune responses. J. Exp. Med. 189: 753-756.
54. Manjili, M. H., R. Henderson, X. Y. Wang, X. Chen, Y. Li, E. Repasky, L. Kazim, and J. R. Subjeck. 2002. Development of a recombinant HSP110-HER-2/neu vaccine using the chaperoning properties of HSP110. Cancer Res. 62: 1737-1742.
55. Ueda, G., Y. Tamura, I. Hirai, K. Kamiguchi, S. Ichimiya, T. Torigoe, H. Hiratsuka, H. Sunakawa, and N. Sato. 2004. Tumor-derived heat shock protein 70-pulsed dendritic cells elicit tumor-specific cytotoxic T lymphocytes (CTLs) and tumor immunity. Cancer Sci. 95: 248-253. References:
56. Koido, S., Tanaka, Y., Tajiri, H., and Gong, J. Generation and functional assessment of antigen-specific T cells stimulated by fusions of dendritic cells and allogeneic breast cancer cells. Vaccine, 25: 2610-2619, 2007.
57. Enomoto, Y., Bharti, A., Khaleque, A. A., Song, B., Liu, C., Apostolopoulos, V., Xing, P. X., Calderwood, S. K., and Gong, J. Enhanced immunogenicity of heat shock protein 70 peptide complexes from dendritic cell-tumor fusion cells. J Immunol, 177: 5946-5955, 2006.

1. A method of inducing a CD4⁺ or CD8⁺ immune reaction to a population of tumor cells in a subject, comprising administering to the subject a chaperone or chaperone protein complex or aggregates thereof, recovered from a heterohybrid cell, wherein the heterohybrid cell is a fusion of at least one antigen presenting cell and at least one tumor cell.

2. The method of claim 1, wherein the heterohybrid cell expresses at least one marker specific to the antigen presenting cell and at least one marker specific to the tumor cell.

3. The method of claim 1, wherein a marker specific for the antigen presenting cell is selected from a group consisting of; CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II.

4. The method of claim 1, wherein a marker specific for the tumor cell is a tumor-associated antigen.

5. The method of claim 1, wherein the marker is selected from a group consisting of; prostate PSA, PSMA, Her-2/neu, HSP-70, EGF-R, MUC-1, Melan-A, MART1, p53, CEA, NYESO, TRP-1, TRP-2, MAGEs, BAGEs, TAG72 or GP-100.

6. The method of claim 1, wherein the heterohybrid cell comprises the genetic material of more than one cell.

7. The method of claim 1, wherein the heterohybrid cell has at least two nuclei.

8. The method of claim 1, wherein the heterohybrid cell is a tetraploid hybrid cell.

9. The method of claim 1, wherein the heterohybrid cell is a bikaryonic or trikaryonic heterohybrid cell.

10. The method of claim 1, wherein the tumor cell is allogenic and the antigen presenting cell is a dendritic cell.

11. The method of claim 1, wherein the chaperone is a heat shock protein.

12. The method of claim 1, wherein the heat shock protein is selected from the group consisting of HSP70, HSP70-1a, HSP701b, HSP70-HOM, HSP70-4, HSP70-5, HSP70-9b, HSP75 or HSC70 or derivatives, isoforms or homologues thereof.

13. The method of claim 11, wherein the heat shock protein is HSP70 or derivatives, isoforms or homologues thereof.

14. The method of claim 11, wherein the heat shock protein is selected from the group consisting of gp96, BiP (Grp78), GrpE, HSP110, HSP90, HSP86, HSP84, HSP78, HSP75, HSP60, HSP40, HSP27, HSP20, α-crystallins, calreticulin or a derivatives, isoforms or homologues thereof.

15. The method of claim 11, wherein the tumor cell is a human tumor cell.

16. The method of claim 1, wherein the subject is a human.

17. The method of claim 1, wherein administration of the chaperone protein or chaperone complex or aggregates thereof reduces tolerance in the subject to a population of tumor cells.

18. A heat shock protein or heat shock protein complex or aggregates thereof recovered from a heterohybrid cell, wherein the heterohybrid cell is a fusion of at least one antigen presenting cell and at least one tumor cell.

19. The heat shock protein or heat shock protein complex of claim 18, wherein the heterohybrid cell expresses at least one marker specific to the antigen presenting cell and at least one marker specific to the tumor cell.

20. The heat shock protein or heat shock protein complex of claim 18, wherein a marker specific to an antigen presenting cell is selected from a group consisting of; CD44; CD64, CD32, CD16, CD89, CD83, CD80, CD86, DC-SIGN, ICAM-1 (CD54); LFA-1, LFA-3 (CD-58), CCR1, CCR2, CCR5, CCR6 and CXCR1, CCR7, TRANCE R/RANK, OX40L, LFA-1, ICAM-1, LFA-3, CD44, ICAM-3, DC-SIGN, CXCR4, MDC/CCL22, TARC/CCL17, PARC/CCL18, CD91, DC-LAMP, B7-1/CD80, B7-2/CD86, CD47, LR3, DORA, ILT-3; DEC-205, DCIR, dectin-2, CLEC-1, MR and MHC Class II.

21. The heat shock protein or heat shock protein complex of claim 18, wherein a marker specific for the tumor cell is a tumor-associated antigen.

22. The heat shock protein or heat shock protein complex of claim 18, wherein the marker selected from a group consisting of; prostate PSA, PSMA, Her-2/neu, HSP-70, EGF-R, MUC-1, Melan-A, MART1, p53, CEA, NYESO, TRP-1, TRP-2, MAGEs, BAGEs, TAG72 or GP-100.

23. (canceled)

24. A composition for the treatment or prevention of cancer in a subject, the composition comprising a heat shock protein or heat shock protein complex or aggregates thereof recovered from a heterohybrid cell according to claim 18.

25. A composition for the treatment or prevention of cancer in a subject, the composition comprising a heat shock protein or heat shock protein complex or aggregates thereof recovered from a heterohybrid cell according to claim 19.

26. A composition for the treatment or prevention of cancer in a subject, the composition comprising a heat shock protein or heat shock protein complex or aggregates thereof recovered from a heterohybrid cell according to claim 20.

27. A composition for the treatment or prevention of cancer in a subject, the composition comprising a heat shock protein or heat shock protein complex or aggregates thereof recovered from a heterohybrid cell according to claim 21.

28. A composition for the treatment or prevention of cancer in a subject, the composition comprising a heat shock protein or heat shock protein complex or aggregates thereof recovered from a heterohybrid cell according to claim 22.


SEQUENCE LISTING (2 sequences)

SEQ ID NO: 1 - protein, 9 aa, Artificial Sequence
  Description of Artificial Sequence Synthetic peptide
DTRPAPGST

SEQ ID NO: 2 - protein, 9 aa, Artificial Sequence
  Description of Artificial Sequence Synthetic peptide
GSTAPPAHG